(19) United States
(12) Patent Application Publication
(10) Pub. No.: US 2024/0410021 A1
(43) Pub. Date: Dec. 12, 2024

(54) PRE-ENRICHMENT FOR SINGLE-CELL ANALYSIS FOR DETECTING MEASUREMENTS OF RESIDUAL DISEASE AND ANALYZING CIRCULATING TUMOR CELLS

(71) Applicant: Mission Bio, Inc., South San Francisco, CA (US)

(72) Inventors: Aaron Thomas Llanso, Olney, MD (US); Adam Sciambi, South San Francisco, CA (US)

(21) Appl. No.: 18/744,163

(22) Filed: Jun. 14, 2024

Related U.S. Application Data
(63) Continuation of application No. PCT/US2022/081869, filed on Dec. 16, 2022.
(60) Provisional application No. 63/290,158, filed on Dec. 16, 2021.

(51) Int. Cl. C12Q 1/6886; C12Q 1/6874; G01N 33/574

(52) U.S. Cl. CPC C12Q 1/6886; C12Q 1/6874; G01N 33/57492; C12Q 2600/156

(57) ABSTRACT

Disclosed herein are methods for analyzing rare disease cells of a plurality of subjects. The methods include obtaining a plurality of samples from the plurality of subjects; for each sample in the plurality of samples, enriching the sample to obtain rare disease cells; pooling the obtained rare disease cells across the plurality of samples; providing the pooled rare disease cells for single-cell analysis to generate amplicons derived from analytes of the pooled rare disease cells; sequencing the amplicons derived from analytes of the pooled rare disease cells; clustering the rare disease cells across the plurality of samples using the sequenced amplicons; and de-multiplexing the rare disease cells by assigning clusters of rare disease cells to individual subjects of the plurality of subjects.

### CROSS REFERENCE TO RELATED APPLICATIONS

This application is a continuation of International (PCT) Application No. PCT/US2022/081869, filed on Dec. 16, 2022, which claims the benefit of and priority to U.S. Provisional Patent Application No. 63/290, 158, filed Dec. 16, 2021, the entire disclosures of each which are hereby incorporated by reference in their entireties for all purposes.

### BACKGROUND

Acute myeloid leukemia (AML) is a heterogeneous set of hematologic malignancies, characterized by expansion of immature myeloid blasts. Although most patients with AML show an initial response to therapy (60-80%), relapse remains the fundamental challenge to achieving durable cures. Measurable residual disease (MRD) represents a critical, therapy-resistant cancer cell reservoir responsible for disease recurrence. Accurately identifying relevant MRD clones is valuable for risk stratifying patients and to guide further therapy to prevent overt relapse and to achieve durable remissions.

### SUMMARY OF THE INVENTION

Disclosed herein are methods for analyzing rare disease cells of a plurality of subjects. In various embodiments, rare disease cells involve cells informative for determining a measurable residual disease (MRD), also referred to herein as a minimal residual disease. In various embodiments, rare disease cells are circulating tumor cells. Generally, methods disclosed herein involve steps of 1) enrichment of rare disease cells, 2) pooling of rare disease cells from various subjects, 3) analyzing the pooled rare disease cells using single-cell analysis techniques, 4) and de-multiplexing the resulting amplicons. Methods disclosed herein can be improved methods for detecting and characterizing rare or residual disease populations within cancer patients. Additionally, methods disclosed herein may be useful as a mainstay clinical diagnostic assay that enables detection and characterization through a test (e.g., invasive or non-invasive test) that yields highly valuable prognostic insight into tumor status: clonal architecture, metastatic potential, therapeutic resistance/susceptibility, characterization of multiple lesions without surgical intervention, etc. This is useful in multiple clinical time points, including baseline diagnostics, therapeutic surveillance (treatment response/efficacy or tumor progression), measurable residual disease detection for patients determined to be in radiographic remission, and identification of persistent or progressive tumor clones.

Disclosed herein is a method for analyzing rare disease cells of a plurality of subjects, the method comprising: obtaining a plurality of samples from the plurality of subjects; for each of one or more samples in the plurality of samples, enriching the sample to obtain rare disease cells; pooling the obtained rare disease cells across the plurality of samples; providing the pooled rare disease cells for single-cell analysis to generate amplicons derived from analytes of the pooled rare disease cells; sequencing the amplicons derived from analytes of the pooled rare disease cells; clustering the rare disease cells across the plurality of samples using the sequenced amplicons; and de-multiplexing the rare disease cells by assigning clusters of rare disease cells to individual subjects of the plurality of subjects.

In various embodiments, enriching the sample comprises performing any of flow cytometry, cell separation, or magnetic bead isolation.

In various embodiments, performing flow cytometry comprises enriching the sample for CD34+ and/or CD117+ cells.

In various embodiments, performing cell separation comprises providing the sample to an Angle Parsotix CTC enrichment platform.

In various embodiments, enriching the sample to obtain rare disease cells further comprises: staining the rare disease cells using one or more oligo-conjugated antibodies, wherein each of the one or more oligo-conjugated antibodies are specific for a protein analyte of the rare disease cells.

In various embodiments, the rare disease cells are circulating tumor cells or cells informative for determining measurable residual disease (MRD).

In various embodiments, the method detects measurable residual disease at a sensitivity better than 0.05%.

In various embodiments, the method detects measurable residual disease at a sensitivity better than 0.01%.

In vairous embodiments, the cells informative for determining MRD are acute myeloid leukemia, myelodysplastic, or myeloid proliferative neoplasm cells.

In various embodiments, for each sample, enriching the sample to obtain rare disease cells comprises obtaining less than 50,000 rare disease cells from the sample.

In various embodiments, for each sample, enriching the sample to obtain rare disease cells comprises obtaining less than 30,000 rare disease cells from the sample.

In various embodiments, for each sample, enriching the sample to obtain rare disease cells comprises obtaining less than 500 rare disease cells from the sample.

In various embodiments, for each sample, enriching the sample to obtain rare disease cells comprises obtaining less than 100 rare disease cells from the sample.

In various embodiments, pooling the obtained rare disease cells across the plurality of samples comprises pooling at least 100,000 rare disease cells.

In various embodiments, analytes of the pooled rare disease cells are one or more of DNA, RNA, or protein analytes.

In various embodiments, analytes of the pooled rare disease cells are RNA analytes.

In various embodiments, clustering the rare disease cells across the plurality of samples using the sequenced amplicons comprises clustering the rare disease cells according to sequenced amplicons derived from the RNA analytes.

In various embodiments, analytes of the pooled rare disease cells comprise both DNA and protein analytes.

In various embodiments, clustering the rare disease cells across the plurality of samples using the sequenced amplicons comprises clustering the rare disease cells according to sequenced amplicons derived from both the DNA and protein analytes.

In various embodiments, the single-cell analysis comprises performing, within a droplet, cell lysis, cell barcoding, and nucleic acid amplification.

In various embodiments, the single-cell analysis comprises performing, cell lysis within a first droplet, and further performing cell barcoding and nucleic acid amplification in a second droplet.

In various embodiments, pooling the obtained rare disease cells across the plurality of samples further comprises incorporating one or more known cells derived from the plurality of subjects.

In various embodiments, assigning clusters of rare disease cells to individual subjects of the plurality of subjects is based on presence of the one or more known cells within the clusters.

Additionally disclosed herein is a system for analyzing rare disease cells of a plurality of subjects, the system comprising: an enrichment platform for enriching a plurality of samples obtained from the plurality of subjects to obtain rare disease cells; a single-cell analysis platform for generating amplicons, wherein the amplicons are derived from analytes of the rare disease cells pooled across the plurality of samples; a sequencing platform for sequencing the amplicons derived from analytes of the pooled rare disease cells; and a computing device for clustering by using the sequenced amplicons and de-multiplexing the rare disease cells by assigning clusters of rare disease cells to individual subjects of the plurality of subjects.

In various embodiments, the enrichment platform is configured to perform any of flow cytometry, cell separation, or magnetic bead isolation.

In various embodiments, the samples are enriched for CD34+ and/or CD117+ cells by using a flow cytometry device.

In various embodiments, the enrichment platform comprises an Angle Parsotix CTC enrichment platform.

In various embodiments, the enrichment platform is configured to stain the rare disease cells using one or more oligo-conjugated antibodies, wherein each of the one or more oligo-conjugated antibodies are specific for a protein analyte of the rare disease cells.

In various embodiments, the rare disease cells are circulating tumor cells or cells informative for determining measurable residual disease (MRD).

In various embodiments, the system detects measurable residual disease at a sensitivity better than 0.05%.

In various embodiments, the system detects measurable residual disease at a sensitivity better than 0.01%.

In various embodiments, the cells informative for determining MRD are acute myeloid leukemia, myelodysplastic, or myeloid proliferative neoplasm cells.

In various embodiments, the enrichment platform enriches the plurality of samples to obtain less than 50,000 rare disease cells.

In various embodiments, the enrichment platform enriches the plurality of samples to obtain less than 30,000 rare disease cells.

In various embodiments, the enrichment platform enriches the plurality of samples to obtain less than 500 rare disease cells.

In various embodiments, the enrichment platform enriches the plurality of samples to obtain less than 100 rare disease cells.

In various embodiments, the pooled rare disease cells comprise at least 100,000 rare disease cells.

In various embodiments, the analytes of the pooled rare disease cells are one or more of DNA, RNA, or protein analytes.

In various embodiments, the analytes of the pooled rare disease cells are RNA analytes.

In various embodiments, the sequenced amplicons are derived from the RNA analytes.

In various embodiments, analytes of the pooled rare disease cells comprise both DNA and protein analytes.

In various embodiments, the sequenced amplicons are derived from both the DNA and protein analytes.

In various embodiments, the single-cell analysis platform is configured to perform, within a droplet, cell lysis, cell barcoding, and nucleic acid amplification.

In various embodiments, the single-cell analysis platform is configured to perform cell lysis within a first droplet, and further perform cell barcoding and nucleic acid amplification in a second droplet.

In various embodiments, the pooled rare disease cells incorporate one or more known cells derived from the plurality of subjects.

In various embodiments, the computing device is configured to assign clusters of rare disease cells to individual subjects of the plurality of subjects based on presence of the one or more known cells within the clusters.

### BRIEF DESCRIPTION OF THE SEVERAL VIEWS OF THE DRAWINGS

These and other features, aspects, and advantages of the present invention will become better understood with regard to the following description, and accompanying drawings, where:

FIGS. 1A and FIG. 1B depict an overall system environment, in accordance with some embodiments.

FIG. 2 is a flow diagram for analyzing rare disease cells of a plurality of subjects, in accordance with an embodiment.

FIG. 3 depicts an example computing device for implementing system and methods described in reference to FIGS. 1A, 1B, and 2.

FIG. 4 depicts an example flow process for analyzing measurable residual disease (MRD) relevant cells.

FIG. 5 shows the improved detection of blasts captured per patient and improved savings/sample when implementing pre-enrichment of cells.

FIG. 6 depicts an example flow process for analyzing circulating tumor cells.

FIGS. 7A-7C depict limit of mutation detection with the scMRD assay. FIG. 7A illustrates schematic of gating strategy for flow cytometric enrichment of live CD34+ and/or CD117+ cells. FIG. 7B illustrates representative heatmap showing mutation calling of spiked-in AML blasts in a limit of detection experiment testing a sensitivity of 0.1%. FIG. 7C illustrates a summary of mutation detection at various sensitivity levels. This plot represents two independent experiments.

FIGS. 8A-8D depict mutation and relapse associated clone identified by scMRD assay. FIG. 8A illustrates Oncoprint showing concordance of MRD detection by bulk NGS assay, scMRD assay and MFC. Bar plot (top) represents the number of cells recovered after computational demultiplexing. FIG. 8B illustrates a representative deconvolution plot of one multiplexed scMRD run. FIG. 8C illustrates comparison of mutations detected by bulk NGS vs scMRD. FIG. 8D illustrates Clonograph of a patient (MRD5-S2) illustrating scMRD-specific detection of NPM1 and JAK2 mutations that were present at late relapse.

FIGS. 9A-9D depict clone-and mutation-specific immunophenotype. FIG. 9A illustrates clone specific immunophenotype. FIG. 9B illustrates differential surface marker expression between CH/preleukemic vs leukemic clones. FIGS. 9C and 9D illustrate UMAP analysis of immunophenotypes of CH/preleukemic vs leukemic clones.

FIGS. 10A-10C depict scDNA+protein analysis that enables simultaneous identification of donor cells and MRD. FIG. 10A illustrates aggregated deconvolution plot showing mutations detected and host-donor chimerism of post-allogeneic HSCT samples included in the study. FIG. 10B illustrates Heatmap analysis of differential surface maker expression between donor and host cells in MRD1-S4.FIG. 10C illustrates concordance of immunophenotype of MRD cells between MFC and scMRD in MRD1-S4.

FIGS. 11A-11F depict workflow and computational demultiplexing of scMRD data. FIG. 11A illustrates schema of scMRD workflow; FIGS. 11B-11F illustrate representative examples of the computational pipeline output.

FIGS. 12A-12E depict deconvolution plots for scMRD runs.

FIGS. 13A-13D depict representative clonographs of MRD samples.

FIGS. 14A-14C depict analysis of protein sequencing data of MRD clones. FIG. 14A illustrates violin plots showing log-normalized differential surface marker expression of various MRD clones. FIG. 14B illustrates violin plots showing log-normalized differential surface marker expression of CH/preleukemic (DNMT3A) vs leukemic (NPM1, DNMT3A/NPM1, DNMT3A/NPM1/FLT3ITD) clones. FIG. 14C illustrates radar plot showing differential surface marker expression of CH/preleukemic (DNMT3A) vs leukemic (DNMT3A/NPM1, DNMT3A/IDH2) clones.

FIGS. 15A and 15B depict concordance of immunophenotype between MFC and scMRD assay from a representative patient (MRD4-S1). FIG. 15A illustrates flow plots showing abnormal expression of bright CD117, dim to negative CD38 and partial CD5 on CD34 positive myeloblasts. FIG. 15B illustrates scMRD data shows similar immunophenotype.

FIGS. 16A-16C depict example results by implementing the methods and systems as described in FIGS. 1A, 1B, and 2-6.

### DETAILED DESCRIPTION

### Definitions

Terms used in the claims and specification are defined as set forth below unless otherwise specified.

The term “about” refers to a ±10% variation from the nominal value unless otherwise indicated or inferred.

The term “acute myeloid leukemia” or “AML” refers to a heterogeneous set of hematologic malignancies, characterized by expansion of immature myeloid blasts.

The terms “measurable residual disease,” “minimal residual disease” or “MRD” are used interchangeably and generally refer to a population of cancer cells that represents a reservoir for disease relapse in cancer malignancies (e.g., in AML).

The term “subject” encompasses a cell, tissue, or organism, human or non-human, whether in vivo, ex vivo, or in vitro, male or female.

The term “sample” can include a single cell or multiple cells or fragments of cells or an aliquot of body fluid, such as a blood sample, taken from a subject, by means including venipuncture, excretion, ejaculation, massage, biopsy, needle aspirate, lavage sample, scraping, surgical incision, or intervention or other means known in the art.

The phrase “rare disease cells” refers to cells that are in low quantity in a sample obtained from a subject. In various embodiments, rare disease cells are in the sample at a concentration of less than 1 in 10² cells. In various embodiments, rare disease cells are in the sample at a concentration of less than 1 in 10³ cells. In various embodiments, rare disease cells are in the sample at a concentration of less than 1 in 10⁴ cells. In various embodiments, rare disease cells are in the sample at a concentration of less than 1 in 10⁵ cells. In various embodiments, rare disease cells are in the sample at a concentration of less than 1 in 10⁶ cells. In various embodiments, rare disease cells are in the sample at a concentration of less than 1 in 10⁷ cells. In various embodiments, rare disease cells are in the sample at a concentration of less than 1 in 10⁸ cells. In various embodiments, rare disease cells are in the sample at a concentration of less than 1 in 10⁹ cells. A first example of rare disease cells include cells informative for determining a measurable residual disease (MRD), also referred to as MRD relevant cells. Another example of rare disease cells include circulating tumor cells.

It must be noted that, as used in the specification, the singular forms “a,” “an” and “the” include plural referents unless the context clearly dictates otherwise.

### Systems for Performing Analysis of Rare Disease Cells

### Overview

Described herein are systems and methods for performing single cell analyses of a plurality of rare disease cells. For example, methods disclosed herein involve a single cell MRD assay by combining enrichment of rare disease cells with integrated single cell DNA sequencing and/or immunophenotyping. Generally, the systems and methods involve performing enrichment of samples to generate rare disease cells that can then be provided for single-cell analysis. In some embodiments, the enrichment is followed by pooling the obtained rare disease cells to generate a sufficient number of rare disease cells. In some embodiments, the single-cell analysis involves generating amplicons derived from analytes of the rare disease cells, and sequencing the amplicons for further analysis such as clustering the rare disease cells and de-multiplexing the rare disease cells. The demultiplexed rare disease cells can be informative for characterizing the cancer patients from whom the rare disease cells were originally obtained. For example, the demultiplexed rare disease cells can be used to determine immunophenotypes of cancer patients, which can be used to guide treatment and/or therapy that may be effective for the particular immunophenotypes.

Advantageously, the systems and methods as described herein can be applied for detecting and characterizing rare or MRD populations from cancer patients, with sensitivities better than about 0.1%, about 0.05%, or about 0.01%.

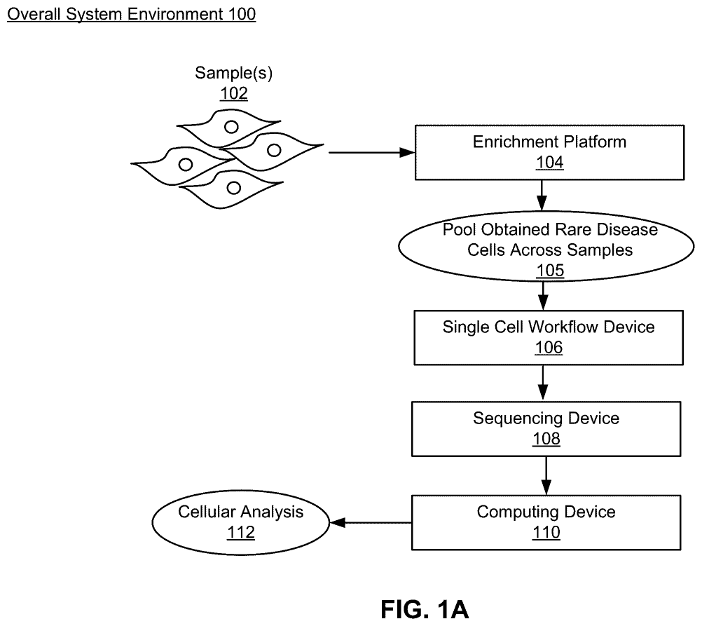

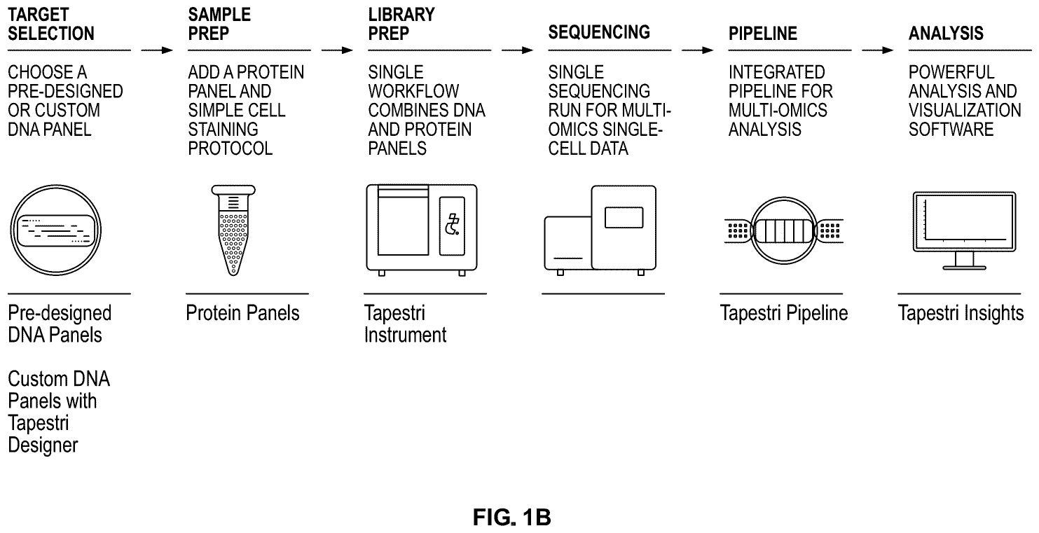

FIGS. 1A and 1B depict an overall system environment, in accordance with some embodiments. In various embodiments, the FIGS. 1A and 1B can include additional or fewer components and/or steps. For example, the step 105 in FIG. 1A need not include all obtained samples 102, and may be based on randomly selected samples. In another example, the single cell analysis as described herein and in FIGS. 1A and 1B may include additional platforms.

Generally, FIG. 1A depicts an overall system environment 100 including an enrichment platform 104, a single cell workflow device 106, a sequencing device 108, and a computing device 110 for analyzing one or more rare disease cells of samples 102, in accordance with some embodiments.

In various embodiments, the samples 102 can be obtained from a subject or a patient. In various embodiments, the samples 102 are healthy cells taken from a healthy subject. In various embodiments, the samples 102 include diseased cells taken from a subject. In one embodiment, the samples 102 include cancer cells taken from a subject previously diagnosed with cancer. For example, cancer cells can be tumor cells available in the bloodstream of the subject diagnosed with cancer. As another example, cancer cells can be cells obtained through a tumor biopsy. Thus, analysis of the tumor cells enables analysis of cells of the subject's cancer. In various embodiments, the samples 102 are obtained from a subject following treatment of the subject (e.g., following a therapy such as cancer therapy). Thus, analysis of the cells enables analysis of cells representing the subject's response to a therapy. In some embodiments, the samples 102 include cancer cells taken from a subject who previously underwent treatment for cancer (e.g., a subject who may be at risk of recurrence). In various embodiments, the samples 102 are or include one or more complete cells. In various embodiments, the samples 102 are or include one or more nuclei and/or partial cells, where the nuclei and/or partial cells are isolated from tissues and/or a suspension of complete cells before the workflow as described herein.

### Enrichment Platform

In general, the enrichment platform 104 may obtain a plurality of samples 102 and may generate rare disease cells from the plurality of samples 102 by enriching the sample. Thus, the rare disease cells are useful for conducting single-cell analysis using the single cell workflow device 106 and for conducting further analysis such as clustering the rare disease cells and de-multiplexing the rare disease cells using the computing device 110. In some embodiments, the rare disease cells provided by the enrichment platform 104 are pooled to generate a pool of samples so that a sufficient number of rare disease cells are generated to provide to the single-cell workflow device 106, sequencing device 108, and computing device 110.

In various embodiments, the enrichment platform 104 enriches cells for rare disease cells, an example of which are CD34+ cells. In various embodiments, the cells are enriched for CD34− cells. In various embodiments, the cells are enriched for CD117+ cells. In various embodiments, the cells are enriched for CD117− cells. In various embodiments, the cells are enriched for CD34+/CD117− populations. In various embodiments, the cells are enriched for CD34+/CD117+ populations. In various embodiments, the cells are enriched for CD34−/CD117− populations. In various embodiments, the cells are enriched for CD34−/CD117+populations.

In various embodiments, the enrichment platform 104 includes flow cytometry, cell separation, and/or magnetic bead isolation instruments to perform enrichment of the samples 102, as described below in further detail. In various embodiments, the enrichment platform 104 includes a flow cytometry instrument. In various embodiments, the enrichment platform 104 includes a cell separation instrument. In various embodiments, the enrichment platform 104 includes a magnetic bead isolation instrument.

In general, flow cytometry includes a lab test to analyze characteristics of cells or particles for obtaining information about the complexities of certain conditions and diseases. The samples needed for performing flow cytometry may include blood, bone marrow, tissue or other body fluid. During the process of flow cytometry, a sample of cells or particles is suspended in fluid and injected into a flow cytometer machine. In some cases, approximately 10,000 cells can be analyzed and processed by a computer in less than one minute.

In general, flow cytometry can be used for cell counting, cell sorting, determining cell function, determining cell characteristics, detecting microorganisms, finding biomarkers, and/or diagnosis and potential treatment of blood and bone marrow cancers. Examples of flow cytometry instruments as used herein include Biolegend instruments.

In general, cell isolation techniques are methods to separate and to transfer certain cells from a complex mixture of cells to obtain single cells or to sort the cells according to a property of choice and thus to generate a more homogenous cell population.

Cell isolation techniques based on flow cytometry may include fluorescence activated cell sorting (FACS) or magnetic activated cell sorting (MACS), which distinguish cells according to their fluorescence or magnetic labeling. Fluorescent dyes and magnetic microbeads can be coupled to cell-type specific antibodies which then allow to uniquely identify target cells from unwanted cells. The cells are then automatically sorted into distinct vials to generate a homogenous cell population for further analyses. These cell isolation techniques offer high throughput cell sorting with little hands-on time.

Application of FACS or MACS cell isolation techniques may use cells in cell suspension form. This is already the case for cells from blood or bone marrow. Many other cell types are embedded in tissue and surrounded by other cell types and extracellular matrix. Thus, tissue blocks can be subjected to mechanical and enzymatic treatment to form a single cell suspension. Collagenases and DNases may be applied to enzymatically digest extracellular matrix proteins and cell-free DNA to ensure that cells are suspended. Cells grown in cell culture can typically be suspended by pipetting or using gentle dissociation buffers.

Advantageously, flow cytometry based cell isolation methods such as FACS or MACS can provide high throughput with little time, and may enable sorting a large number (e.g., thousands) of cells at a time.

In various embodiments, cell isolation techniques are also based on droplet-based methods and enable sorting of cells and combining cell isolation with video documentation of each single cell or with PCR methods for single cell analysis, and thus allow for a combined workflow of single cell isolation and single cell analysis.

In various embodiments, the samples 102 (e.g. cells) are thawed, washed with FACS buffer, and quantified using a cell counter included in the enrichment platform 104. In some embodiments, the cell counter includes a commercially available cell counter such as a Countess cell counter. In various embodiments, the enrichment platform provides an output of about 0.5×10⁶−4.0×10⁶ viable cells for further processing. In various embodiments, the enrichment platform provides an output of about 1.0×10⁶−3.5×10⁶ viable cells may be provided for further processing. In various embodiments, the enrichment platform provides an output of about 1.5×10⁶−3.0×10⁶ viable cells may be provided for further processing. In various embodiments, the enrichment platform provides an output of about 2.0×10⁶−2.5×10⁶ viable cells may be provided for further processing. In various embodiments, the enrichment platform provides an output of about 0.5×10⁶, 1×10⁶, 1.5×10⁶, 2×10⁶, 2.5×10⁶, 3×10⁶, 3.5×10⁶, 4×10⁶ viable cells for further processing.

In various embodiments, a pool of oligo-conjugated antibodies are added and incubated for an additional period of time. In some embodiments, antibodies are specific for cell surface proteins, examples of which include CD4, CD8, CD34, CD117, and CD45. In particular embodiments, TotalSeq™-D Human Heme Oncology Cocktail, V1.0 (#399906, BioLegend) is implemented. In particular embodiments, a pool of 45 oligo-conjugated antibodies are added and incubated for an additional 30 minutes on ice.

In various embodiments, the samples 102 are then washed (e.g., 3 times with cell staining buffer (e.g., #420201, BioLegend)), followed by resuspension of the cells (e.g., in DAPI containing FACS buffer). In particular embodiments, the DAPI negative and CD45 positive viable cells are gated.

In particular embodiments, exclusion of CD4 and CD8 positive lymphocytes is performed.

In various embodiments, after exclusion of CD4 and CD8 positive lymphocytes, CD34+/CD117−, CD34+/CD117+and CD34−/CD117+populations are combined for sorting, e.g., using a SH800S Cell Sorter.

### Single Cell Analysis Platform

The single cell workflow device 106 refers to a device that processes individuals cells to generate amplicons for sequencing. In various embodiments, the single cell workflow device 106 can encapsulate individual cells into a first droplet, lyse cells within the first droplet, perform cell barcoding of cell lysate in a second droplet, and generate amplicons in the second droplet. Thus, amplicons can be collected and sequenced. In various embodiments, the single cell workflow device 106 further includes or provides amplicons to a sequencing device 108 for sequencing the amplicons. In various embodiments, at least 10, 50, 100, 150, 20, 250, 300, 350, 400, 450, or 500 amplicons (e.g., DNA amplicons, RNA amplicons, and/or amplicons derived from antibody oligonucleotides) are generated in a workflow.

Reference is now made to FIG. 1B, which depicts a single-cell analysis workflow including the designing of a targeted panel (e.g., targeted DNA panel), sample preparation (which includes adding a protein panel and/or cell staining protocol), library preparation, cell sequencing, multi-omic analysis, and software analysis. In various embodiments, the single cell workflow device may be a device that performs the “Library Prep” step shown in FIG. 1B. The single cell workflow device may perform steps involving encapsulating and lysing cells in droplets, performing nucleic acid amplification in droplets, and sequencing amplicons. Further details of such a single-cell workflow is described in U.S. Pat. No. 10,161,007, US20220325357, and WO2021/067966, each of which is hereby incorporated by reference in its entirety.

In various embodiments, the single cell analysis as described herein is performed on a Tapestri® workflow instrument or platform. In various embodiments, the single cell analysis as described herein is performed on a 10× Genomics Chromium® platform, or other suitable platforms.

### Sequencing Platform and Read Alignment

Amplified nucleic acids (e.g., amplicons) may be sequenced to obtain sequence reads for generating a sequencing library. Sequence reads can be achieved with commercially available next generation sequencing (NGS) platforms, including platforms that perform any of sequencing by synthesis, sequencing by ligation, pyrosequencing, using reversible terminator chemistry, using phospholinked fluorescent nucleotides, or real-time sequencing. For example, amplified nucleic acids may be sequenced on an Illumina® platform (e.g., Illumina MiSeq platform). In another example, amplified nucleic acids may be sequenced using SOLID technology, HeliScope.

Details for sequencing using the Illumina platform are found in Voelkerding et al., Clinical Chem., 55:641-658, 2009; MacLean et al., Nature Rev. Microbiol., 7:287-296; U.S. Pat. No. 6,833,246; U.S. Pat. No. 7,115,400; U.S. Pat. No. 6,969,488; each of which is hereby incorporated by reference in its entirety. Details for sequencing using SOLID technology are found in Voelkerding et al., Clinical Chem., 55:641-658, 2009; MacLean et al., Nature Rev. Microbiol., 7:287-296; U.S. Pat. No. 5,912,148; U.S. Pat. No. 6,130,073; each of which is incorporated by reference in its entirety. Details for performing sequencing using HeliScope are found in Voelkerding et al., Clinical Chem., 55:641-658, 2009; MacLean et al., Nature Rev. Microbiol., 7:287-296; U.S. Pat. No. 7,169,560; U.S. Pat. No. 7,282,337; U.S. Pat. No. 7,482,120; U.S. Pat. No. 7,501,245; U.S. Pat. No. 6,818,395; U.S. Pat. No. 6,911,345; U.S. Pat. No. 7,501,245; each of which is incorporated by reference in its entirety. Additional details for performing sequencing are found in Margulies et al. (2005) Nature 437:376-380, which is hereby incorporated by reference in its entirety.

Further details for aligning sequence reads to reference sequences is described in U.S. application Ser. No. 16/279,315, which is hereby incorporated by reference in its entirety. In various embodiments, an output file having SAM (sequence alignment map) format or BAM (binary alignment map) format may be generated and output for subsequent analysis, such as for determining cell trajectory.

### Computing Platform

The computing device 110 is configured to receive the sequenced reads from the sequencing device 108. In various embodiments, the computing device 110 is communicatively coupled to the single cell workflow device 106 or the sequencing device 108 and therefore, directly receives the sequence reads from the single cell workflow device 106 or the sequencing device 108. The computing device 110 analyzes the sequence reads to generate a cellular analysis 112.

In some embodiments, the computing device 110 includes components to perform scMRD computational demultiplexing. In various embodiments, the computing device 110 performs computational demultiplexing of rare disease cells by clustering the rare disease cells. In various embodiments, the computing device 110 clusters the rare disease cells according to genomic sequences, such as one or more of single nucleotide polymorphisms (SNPs), single nucleotide variants (SNVs) and/or copy number variants or variations (CNVs). In particular embodiments, deconvolution or demultiplexing of multiplexed scMRD runs involves analyzing presence of germline SNPs. Suspected SNPs may be verified via referencing the Ensembl SNP database through the BioMart R package and may be tallied for non-missing genotyping information within the filtered NGT matrix. In various embodiments, the SNPs present in patients may include NRAS.G12D, RUNX1.P247fs, DNMT3A.F751fs, JAK2. V617F, IDH2.R140Q, IDH2.R140Q, CHEK2.T387I, and/or TET2.P1723S.

In various embodiments, clustering the rare disease cells comprises performing a dimensionality reduction analysis selected from any of principal component analysis (PCA), linear discriminant analysis (LDA), K-means clustering, T-distributed stochastic neighbor embedding (t-SNE), or uniform manifold approximation and projection (UMAP). In particular embodiments, K-means clustering is performed on SNP allele frequencies in a subset of cells with complete SNP genotypes. In various embodiments, the number of clusters for partitioning is set equal to a number of unique patient samples in a given multiplex.

In various embodiments, doublet identification and exclusion may be conducted by first evenly sampling cells from all clusters to form a pool of cells with equal representation of each cluster. Artificial doublets may be then generated via sampling the cell pool two cells at random and averaging the SNP profiles until the proportion of artificial doublets approached 5-10% of the total number of cells in the dataset. Doublets may be then merged with real cells and re-clustered to produce real and artificial cluster centers. The Euclidean distance may be then measured between each real cell and 1) it's respective cluster, 2) the artificial cluster center. The distribution of distances between 90-95% of cells to their respective cluster centers may be used as a cutoff to exclude cells which are within this distance to the artificial cluster center. This process may be repeated 10 times, with random replacement of NA values with allele frequencies of 0, 50, or 100, and cells were excluded if their distance was within the doublet gate in all replicates. After removing doublets and low-quality cells with high similarity to artificial doublets, the most common SNP profile was tallied for each cluster. To classify additional cells, a Hamming distance was calculated between all cells and each SNP profile, without penalizing SNPs with missing genotypes. Cells were assigned to clusters based on matching 80% of the SNP profile and being the maximum Hamming distance from every other cluster. For some multiplexed runs, slightly less stringent filters were applied to reduce the Hamming distance between clusters. After cell classification, each cluster was queried for pathogenic mutations detected by bulk NGS at the diagnosis, remission, and relapse (if applicable) timepoints, and the cell number per cluster was tallied.

In various embodiments, the computing platform includes components to perform single cell protein analysis for each demultiplexed sample, where single cell protein data may be extracted as raw counts. For example, given the demultiplexed cells that have been determined to originate from patients, each demultiplexed patient sample can be analyzed independently for clonality of mutations and clone-specific immunophenotype. Different cellular immunophenotypes can be characterized by differential expression of various immune-related proteins, examples of which include CD34, CD117, CD33, and CD71.

### Methods for Performing Analysis of Rare Disease Cells

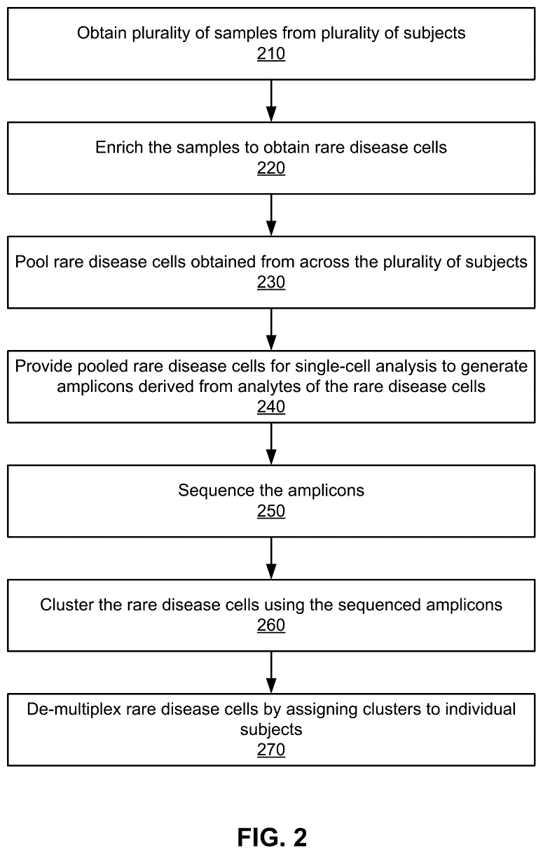

FIG. 2 is a flow diagram for analyzing rare disease cells of a plurality of subjects, in accordance with an embodiment. Generally, the process for analyzing rare disease cells of a plurality of subjects includes the steps 210-270, as shown in FIG. 2.

At step 210, a plurality of samples are obtained from a plurality of subjects. In various embodiments, one sample is obtained from one subject. In various embodiments, a sample is a blood sample. In particular embodiments, a sample includes rare disease cells, such as circulating tumor cells or MRD relevant cells.

At step 220, the samples are enriched to obtain rare disease cells. In various embodiments, each individual sample undergoes enrichment processes to obtain rare disease cells from that individual sample. In various embodiments, the enrichment process includes performing any one of flow cytometry, cell separation, or magnetic bead isolation. For example, for flow cytometry, the sample can be labeled (e.g., using antibodies or fluorescent dye) that can be used to sort rare disease cells from other non-diseased cells (e.g., healthy cells). As another example, performing cell separation can involve providing the sample to an Angle Parsortix circulating tumor cell (CTC) enrichment platform. Thus, CTCs can be enriched in or separated from non-CTCs.

At step 230, rare disease cells obtained from across the plurality of subjects are pooled. Given that there may be limited numbers of rare disease cells that are obtained from a single sample (e.g., at step 220), pooling the rare disease cells obtained across the plurality of subjects generates a sufficient number of cells that can then be provided for single-cell analysis. In various embodiments, pooling the rare disease cells comprises pooling at least 10,000 rare disease cells. In various embodiments, pooling the rare disease cells comprises pooling at least 20,000 rare disease cells. In various embodiments, pooling the rare disease cells comprises pooling at least 30,000 rare disease cells. In various embodiments, pooling the rare disease cells comprises pooling at least 40,000 rare disease cells. In various embodiments, pooling the rare disease cells comprises pooling at least 50,000 rare disease cells. In various embodiments, pooling the rare disease cells comprises pooling at least 60,000 rare disease cells. In various embodiments, pooling the rare disease cells comprises pooling at least 70,000 rare disease cells. In various embodiments, pooling the rare disease cells comprises pooling at least 80,000 rare disease cells. In various embodiments, pooling the rare disease cells comprises pooling at least 90,000 rare disease cells. In various embodiments, pooling the rare disease cells comprises pooling at least 100,000 rare disease cells. In various embodiments, pooling the rare disease cells comprises pooling at least 150,000 rare disease cells. In various embodiments, pooling the rare disease cells comprises pooling at least 200,000 rare disease cells. In various embodiments, pooling the rare disease cells comprises pooling at least 300,000 rare disease cells. In various embodiments, step 230 further involves incorporating one or more known cells that are derived from the plurality of subjects. Known cells refer to cells whose genotype and/or phenotype are known. For example, a known cell can include a cell with known mutations, single nucleotide variants (SNVs) and/or copy number variations (CNVs). As another example, a known cell can include a cell with known expression or non-expression of certain proteins. As another example, a known cell can include a cell with known mutations, SNVs, CNVs, and known expression or non-expression of certain proteins. Incorporating one or more known cells enables the labeling of clusters (e.g., at step 270), which is described further below.

Step 240 involves providing the pooled rare disease cells for single cell analysis. In various embodiments, step 240 involves providing the pooled rare disease cells to a Tapestri® workflow instrument. In various embodiments, the single-cell analysis at step 240 comprises performing, within a droplet, cell lysis, cell barcoding, and nucleic acid amplification. In various embodiments, the single-cell analysis comprises performing, cell lysis within a first droplet, and further performing cell barcoding and nucleic acid amplification in a second droplet. As a result of the single-cell analysis, a plurality of amplicons are generated, wherein the amplicons are derived from analytes of the rare disease cells. In various embodiments, the analytes are any one of DNA, RNA, or protein analytes. In particular embodiments, the analytes are RNA analytes. In particular embodiments, the analytes are DNA analytes. In particular embodiments, the analytes are protein analytes. In particular embodiments, the analytes are both DNA and protein analytes.

Step 250 involves sequencing the amplicons. In various embodiments, step 250 involves performing next generation sequencing. Here, the sequenced amplicons can be aligned to a reference library to determine the sequences (e.g., genomic or transcriptomic sequences) that are present in rare disease cells. In various embodiments, sequencing the amplicons comprises sequencing cell barcodes that are present in the amplicons, thereby enabling the identification or the cellular origin of the amplicons.

Step 260 involves clustering the rare disease cells using the sequenced amplicons of the rare disease cells. In various embodiments, clustering the rare disease cells comprises clustering the rare disease cells according to determined presence or absence of protein analytes. In various embodiments, clustering the rare disease cells comprises clustering the rare disease cells according to determined genomic sequences, such as presence or absence of mutations, single nucleotide variants (SNVs), copy number variations (CNVs), and the like. In particular embodiments, clustering the rare disease cells comprises clustering the rare disease cells according to single nucleotide polymorphisms (SNPs). In various embodiments, clustering the rare disease cells comprises performing a dimensionality reduction analysis selected from any of principal component analysis (PCA), linear discriminant analysis (LDA). T-distributed stochastic neighbor embedding (t-SNE), or uniform manifold approximation and projection (UMAP).

Step 270 involves de-multiplexing rare disease cells by assigning clusters to individual subjects. Thus, step 270 enables the identification of the origin of the rare disease cells. In various embodiments, a cluster is assigned to an individual subject based on the presence of known cells that were incorporated into the pooled rare disease cells (e.g., at step 230). For example, a known cell may be a cell of known protein expression (e.g., an immune cell such as a CD4 T cell or CD8 T cell). Thus, if the known cell is located in a particular cluster, then rare disease cells in the particular cluster can be assigned to the individual subject corresponding to the known cell.

### Computer Embodiments

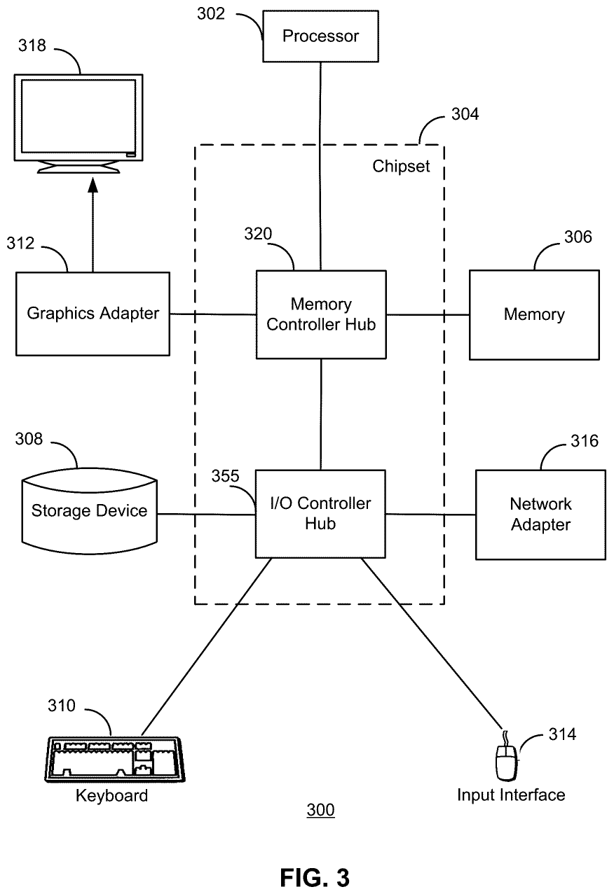

FIG. 3 depicts an example computing device for implementing system and methods described in reference to FIGS. 1-2. In various embodiments, the example computing device 300 serves as the computing device 110 as described in FIG. 1 and the flow diagram shown in FIG. 2. Examples of a computing device can include a personal computer, desktop computer, laptop, server computer, a computing node within a cluster, message processors, hand-held devices, multi-processor systems, microprocessor-based or programmable consumer electronics, network PCs, minicomputers, mainframe computers, mobile telephones, PDAS, tablets, pagers, routers, switches, and the like.

As shown in FIG. 3, in some embodiments, the computing device 300 includes at least one processor 302 coupled to a chipset 304. The chipset 304 includes a memory controller hub 320 and an input/output (I/O) controller hub 355. A memory 306 and a graphics adapter 312 are coupled to the memory controller hub 320, and a display 318 is coupled to the graphics adapter 312. A storage device 308, an input interface 314, and network adapter 316 are coupled to the I/O controller hub 355. Other embodiments of the computing device 300 have different architectures.

The storage device 308 is a non-transitory computer-readable storage medium such as a hard drive, compact disk read-only memory (CD-ROM), DVD, or a solid-state memory device. The memory 306 holds instructions and data used by the processor 302. The input interface is a touch interface, examples of which can be a touch-screen interface, a mouse (e.g., input interface 314), track ball, or other type of input interface, a keyboard (e.g., keyboard 310), or some combination thereof, and is used to input data into the computing device 300. In some embodiments, the computing device 300 may be configured to receive input (e.g., commands) from the input interface via gestures from the user. The graphics adapter 312 displays images and other information on the display 318. The network adapter 316 couples the computing device 300 to one or more computer networks.

The computing device 300 is adapted to execute computer program modules for providing functionality described herein. As used herein, the term “module” refers to computer program logic used to provide the specified functionality. Thus, a module can be implemented in hardware, firmware, and/or software. In one embodiment, program modules are stored on the storage device 308, loaded into the memory 306, and executed by the processor 302.

The types of computing devices 300 can vary from the embodiments described herein. For example, the computing device 300 can lack some of the components described above, such as graphics adapters 312, input interface 314, and displays 318. In some embodiments, a computing device 300 can include a processor 302 for executing instructions stored on a memory 306.

Methods described herein can be implemented in hardware or software, or a combination of both. In one embodiment, a non-transitory machine-readable storage medium, such as one described above, is provided, the medium comprising a data storage material encoded with machine readable data which, when using a machine programmed with instructions for using said data, is capable of executing instructions for analyzing rare disease cells, as described herein. Embodiments of the methods described above can be implemented in computer programs executing on programmable computers, comprising a processor, a data storage system (including volatile and non-volatile memory and/or storage elements), a graphics adapter, an input interface, a network adapter, at least one input device, and at least one output device. A display is coupled to the graphics adapter. Program code is applied to input data to perform the functions described above and generate output information. The output information is applied to one or more output devices, in known fashion. The computer can be, for example, a personal computer, microcomputer, or workstation of conventional design.

Each program can be implemented in a high-level procedural or object-oriented programming language to communicate with a computer system. However, the programs can be implemented in assembly or machine language, if desired. In any case, the language can be a compiled or interpreted language. Each such computer program is preferably stored on a storage media or device (e.g., ROM or magnetic diskette) readable by a general or special purpose programmable computer, for configuring and operating the computer when the storage media or device is read by the computer to perform the procedures described herein. The system can also be implemented as a computer-readable storage medium, configured with a computer program, where the storage medium so configured causes a computer to operate in a specific and predefined manner to perform the functions described herein.

The signature patterns and databases thereof can be provided in a variety of media to facilitate their use. “Media” refers to a manufacture that contains the signature pattern information of the present invention. The databases of the present invention can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure can be chosen, based on the means used to access the stored information. A variety of data processor programs and formats can be used for storage, e.g. word processing text file, database format, etc.

### EXAMPLES

Below are examples of specific embodiments for carrying out the present invention. The examples are offered for illustrative purposes only and are not intended to limit the scope of the present invention in any way. Efforts have been made to ensure accuracy with respect to numbers used (e.g., amounts, temperatures, etc.), but some experimental error and deviation should be allowed for.

### Example 1

### Example Method for Analyzing MRD-Relevant Cells

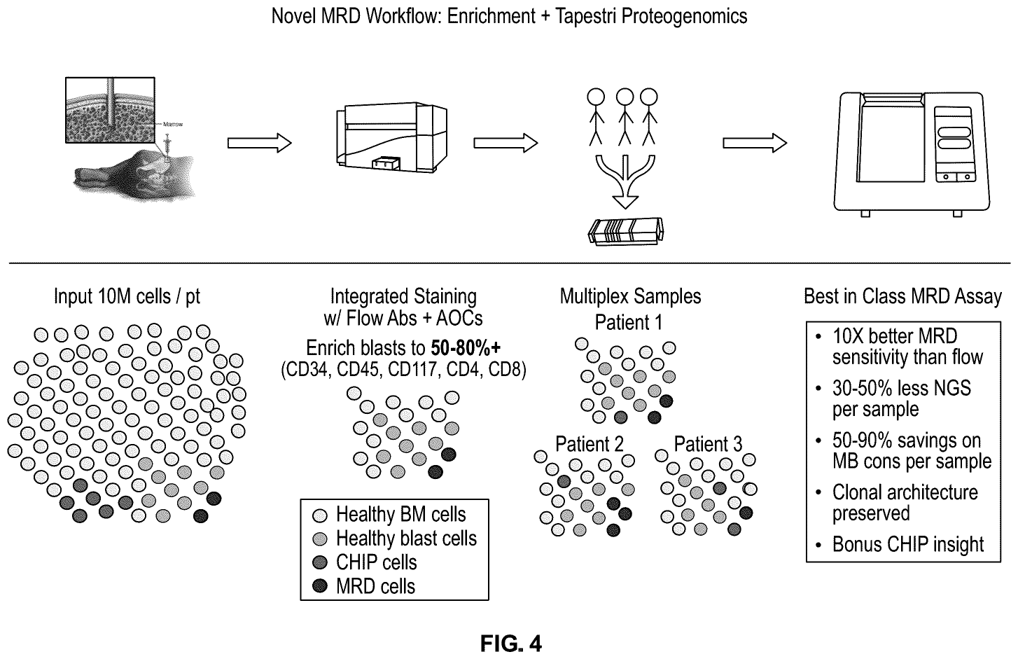

FIG. 4 depicts an example flow process for analyzing measurable residual disease (MRD) relevant cells. This technique is a novel protocol that leverages population enrichment techniques (e.g. flow cytometry or immune-magnetic bead-based technologies) upstream of the multi-omic (DNA+Protein) Tapestri assay, thereby enabling increased detection sensitivity relative to selected (enriched) populations within a clonally heterogeneous sample. Because population enrichment often yields insufficient total cell numbers for optimal input into the Tapestri cartridge, a sample multiplexing approach may be useful in order to pool multiple samples from different patients to ultimately achieve an optimal number of cells for input to the single-cell multi-omics assay. In this case, the germline genetic diversity observed in patient samples is leveraged to demultiplex pooled samples after next-generation sequencing.

The integration and sequence of these protocols enables consolidation of duplicative antibody staining and washing steps used independently for both flow cytometry and TotalSeq-D immunophenotype staining. These stain/wash steps are consolidated into a single stain/wash protocol that includes both the flow cytometric (fluorochrome conjugated)—or other—antibodies and the TotalSeq-D immunophenotyping antibodies (Antibody-oligo-conjugates) in the same reaction. Distinct, non-interfering antibody clones are selected to avoid steric hinderance of antibodies binding the same target protein for purposes of both enrichment and immunophenotypic characterization.

Four problems are solved with this approach, specific to Measurable Residual Disease (MRD) detection.

1) Assay Sensitivity: Limit of Detection (LOD) expected to be at least 1 order of magnitude greater than current clinical standard of care for MRD detection in AML (Flow cytometry). Current LOD standard as determined by the European LeukemiaNet MRD Working Party is 0.1% for flow cytometry detection of residual AML. The Tapestri workflow will achieve 0.01% detection sensitivity.

2) Specificity/Accuracy: Integration of DNA and immunophenotype

information at single-cell resolution enables the novel capability to distinguish, in one clinical example, CHIP (benign) cells that confound accurate detection of residual disease (reduces false positives and false negatives), as the mutations frequently observed in CHIP are also implicated as initiating events in malignancy. As such, these mutant cells cannot be readily distinguished as benign or residual cancer without the multi-omic genotype and cell state/fate information (Tapestri DNA+Protein assay).

3) Clonal Architecture: Resolves clonal heterogeneity of persisting disease populations with important clinical and therapeutic implications. Bulk next generation sequencing (Bulk NGS) approaches currently in use only generate variant allele frequency information and do not resolve variant co-occurrence or enable conclusive clonal and sub-clonal disease analysis, which are demonstrated in the literature to yield considerable prognostic & clinical value.

4) Cost: Enrichment efficiency and sample multiplexing of multiple patient samples reduces the assay cost between 50-90% (depending on number of samples in multiplex), thereby delivering unprecedented multi-omic single-cell resolution insight into disease state of rare and enriched populations at reasonable price points similar to existing bulk NGS clinical assays.

The germline patient genotype-derived sample multiplexing strategy accomplishes multiple key improvements:
Improves assay sequencing efficiency (5% to >80% NGS reads allocated to target populations)
Enables improved assay sensitivity by virtue of accommodating low-input enriched samples
Scalability, accommodating multiple samples per run
Reduces total assay cost (50-80%) by adding more denominator samples to the cost of a single cartridge.
Does not require any additional sample manipulation (e.g. antibody or oligo hashing protocols), provided appropriate prior sequencing data is available for each patient. This is usually obtained from intake diagnostic sequencing tests.

This is an approach that combines flow cytometric-based population enrichment with downstream high-throughput single cell multi-omic analysis for unprecedented resolution into residual disease biology. Here, antibody staining workflows (flow and Tapestri AOC staining) are combined into a single stain/wash protocol with carefully selected antibody clones for each technique. This approach entails significant loss (˜50% +) of the sample, thereby reducing assay sensitivity. However, in this workflow, the requisite washing steps are consolidated rather than duplicated, and this is based on the fact that the flow cytometer serves as an effective washing strategy in itself, eliminating need for additional downstream washes. This markedly reduces cell loss. The sample is stained with both flow cytometry and AOC antibodies, then is enriched for specific cellular populations by flow cytometry. The resulting enriched cells are loaded directly on to the Tapestri platform. In many disease states, the enriched fraction of cells is insufficient for input on the Tapestri cartridge, which is then addressed by pooling multiple enriched patient samples into a single sample that meets minimum input requirements. These patient samples are readily de-multiplexed using unique germline variants specific to each patient. This genotype-derived multiplexing strategy is valuable for future clinical considerations downstream as this both increases cell capture efficiency and reduces cost of the assay, thereby paving the way for realistic reimbursement considerations in the future.

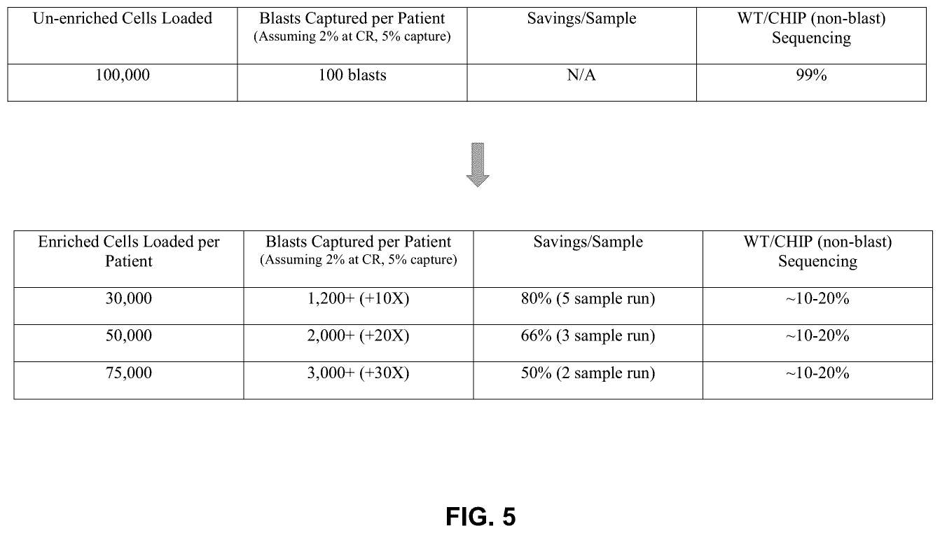

FIG. 5 depicts the improvement conferred by methods disclosed herein including the pre-enrichment of cells followed by single-cell analysis. Notably, FIG. 5 shows the number of cells loaded per patient following enrichment or non-enrichment, number of blasts captured per patient, the savings per sample, and percentage of wild type (WT) non-blast cells that are sequenced. As shown in FIG. 5, following enrichment of cells, 30,000 cells, 50,000 cells, or 75,000 cells per patient were loaded on Tapestri. Notably, the number of cells loaded here following pre-enrichment are less than the number of cells that were loaded when pre-enrichment was not performed. The number of blasts captured per patient as a result of pre-enrichment (e.g., 1,200 for 30,000 loaded cells, 2,000 for 50,000 loaded cells, and 3,000 for 75,000 loaded cells) was significantly increased in comparison to the number of blasts captured per patients when pre-enrichment was not performed, even despite the higher number un-enriched cells that were loaded. Furthermore, pre-enrichment resulted in significant savings per sample. Finally, pre-enrichment resulted in a significant decrease in the number of wild type (WT) non-blast cells that were sequenced.

### Example 2

### Example Method for Analyzing Circulating Tumor Cells

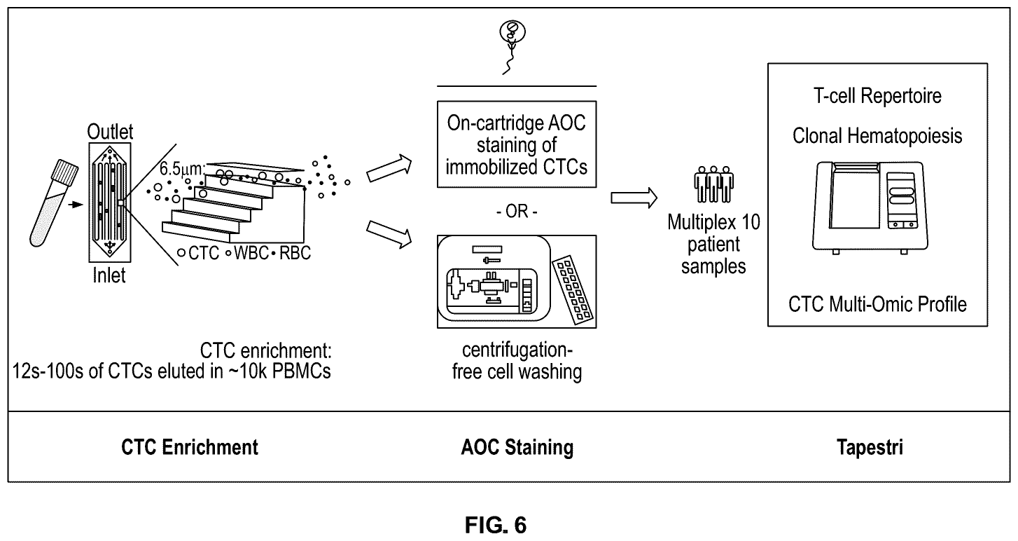

FIG. 6 depicts an example flow process for analyzing circulating tumor cells. This novel workflow combines two distinct platforms including the Angle Parsortix CTC enrichment platform located upstream of the Tapestri single-cell multi-omics analysis platform. The Angle Parsortix instrument takes whole blood as input, and using physical attributes of the circulating tumor cells (i.e. weight, size, etc.), enables enrichment of these rare tumor cells into a carrier population of peripheral blood mononuclear cells (PBMCs). This enriched output is not pure CTCs, which is arguably less valuable than other technical approaches that yield pure CTCs captured for profiling. However, this PBMC populations of cells is advantageous for the combination with Tapestri, as it acts as a “carrier” population. An appropriate input into the Tapestri system is 100,000 cells that are to be loaded into the cartridge, and since most CTC capture platforms yield low numbers of tumor cells (five to low hundreds), there is a large gap between typical CTC recovery number and the input specifications for the Tapestri platform. Furthermore, for integrated multi-omic profiling, (i.e. DNA and protein characterization), there is a recommended input of 1,000,000 cells for the Tapestri assay. This is of course a major gap between typically expected total CTC numbers and input requirements. Additionally, there is considerably cell loss introduced in the Tapestri antibody-staining protocol, further complicating feasibility with loss of highly precious and rare CTCs. The Angle Parsortix platform enables antibody-staining of captured CTC populations on the cartridge in a protocol with little or no loss to the CTC population. This enables staining of the CTC population with the Tapestri-specific antibody-oligo-conjugates (AOCs) on the Angle Parsortix platform. The now antibody-labeled CTC population is eluted from the Angle Parsortix cartridge in a carrier population of the PBMCs. The resulting sample is then pooled with other patient-distinct samples processed in the same workflow for multiplex processing on a single cartridge. This multiplexing approach enables attainment of a target cell input (100,000 cells) on the Tapestri cartridge, while yielding dramatic cost reduction on a per-sample basis. The multiplexing strategy is unique in that patient-distinct profiles of germline single-nucleotide polymorphisms enable reliable de-multiplexing without additional sample modification. The final workflow is a novel amalgum of both Tapestri and Angle Parsortix technologies that uniquely enable multi-omic characterization of rare circulating tumor cells.

### Example 3

### Example Methods and Systems for Analyzing MRD-Relevant Cells

MRD serves as a reservoir for disease relapse in acute myeloid leukemia (AML) and other malignancies. Understanding the biology enabling MRD clones to resist therapy is valuable to guide the development of more effective curative treatments. Discriminating between residual leukemic clones, preleukemic clones and normal precursors remains a challenge with traditional MRD tools.

In this example, a single cell MRD assay was developed to resolve challenges associated with bulk next generation sequencing and multi-color flow cytometry (MFC) MRD-testing, by combining flow cytometric enrichment of the targeted precursor/blast population with integrated single cell DNA sequencing and immunophenotyping.

Advantageously, the single cell MRD assay as described herein showed improved performance as compared with traditional MRD tools (e.g., bulk next generation sequencing and MFC MRD-testing), and thus may enhance MRD detection while simultaneously illuminating the clonal architecture of clonal hematopoiesis/pre-leukemic and leukemic cells surviving AML therapy.

### 3.1 Methods and Systems

### 3.1.1 Patient Samples

Bone marrow aspirates were received in a clinical lab. After 5 days with clinical tests being completed, the leftover cells were deemed as medical waste and mononuclear cells were obtained by centrifugation on Ficoll from bone marrow and viably frozen. Uninvolved bone marrow aspirates from patients with stage 1 B-cell lymphoma were used as normal controls. Patient samples underwent high-throughput genetic sequencing with an FDA approved targeted deep sequencing assay of 500 genes (IMPACT-heme) or by an NGS platform panel composed of 49 genes that are recurrently mutated in myeloid disorders (RainDance Technologies ThunderBolts Myeloid Panel).

### 3.1.2 Cell Enrichment

Patient samples were thawed, washed with FACS buffer, and quantified using a Countess cell counter. Cells (0.5−4.0×10⁶ viable cells) were then resuspended in cell staining buffer (#420201, BioLegend) and incubated with TruStain FcX, and 1× Tapestri blocking buffer for 15 min on ice. Cells were incubated with anti-human CD4 (clone: OKT4)-APC/Cy7 (dilution 1:30), anti-human CD8 (clone: RPA-T8)-BV711 (dilution 1:30), anti-human CD34 (clone: Qbend)-APC (dilution 1:10), anti-human CD117 (clone: A3C6E2)-PE (dilution 1:75), and anti-human CD45 (clone: Q17A19)-AlexaFlour 488 (dilution 1:30) for 15 minutes on ice. Then TotalSeq™-D Human Heme Oncology Cocktail, V1.0 (#399906, BioLegend) containing the pool of 45 oligoconjugated antibodies was added and incubated for an additional 30 minutes on ice. Cells were then washed 3 times with cell staining buffer (#420201, BioLegend) followed by resuspension of the cells in DAPI containing FACS buffer. DAPI negative and CD45 positive viable cells were gated. After exclusion of CD4 and CD8 positive lymphocytes, CD34+/CD117−,CD34+/CD117+ and CD34−/CD117+populations were combined for sorting using a SH800S Cell Sorter. In MRD1 run, 1000 sorted CD4 vs CD8 positive T-cells from two individual samples were spiked in, respectively.

### 3.1.3 Single-Cell DNA and Protein Library Preparation and Sequencing

Enriched cells were resuspended in Tapestri cell buffer and quantified using a Countess cell counter (Invitrogen). Single cells (1,000-3,000 cells/μl) were encapsulated using a Tapestri microfluidics cartridge and lysed. A forward primer mix (30 μM each) for the antibody tags was added before barcoding. Barcoded samples were then subjected to targeted PCR amplification of a custom 109 amplicons covering 31 genes known to be involved in AML. DNA PCR products were then isolated from individual droplets and purified with Ampure XP beads. The DNA PCR products were then used as a PCR template for library generation as above and repurified using Ampure XP beads. Protein PCR products (supernatant from Ampure XP bead incubation) were incubated with Tapestri pullout oligo (5 μM) at 96° C. for 5 min followed by incubation on ice for 5 min. Protein PCR products were then purified using Streptavidin C1 beads (Invitrogen) and beads were used as a PCR template for the incorporation of i5/i7 Illumina indices followed by purification using Ampure XP beads. All libraries, both DNA and protein, were quantified using an Agilent Bioanalyzer and pooled for sequencing on an Illumina NovaSeq.

### 3.1.4 Data Processing and Variant Filtering

FASTQ files from single cell DNA+protein samples were processed via the TapestriV2 pipeline, an analytics platform to trim adaptor sequences, align sequencing reads to the hg19 reference genome, and call cells based on completeness of amplicon sequencing reads for each barcode, and call variants using GATKv3.7 best practices. After pipeline processing, data for each run were aggregated into H5 files, which were downloaded and read into R using the rhdf5 package. Downstream processing was conducted using custom scripts in R (https://github.com/RobinsonTroy/single cell MRD). Low quality variants and cells were then excluded based on filtering cutoffs for genotype quality score (<30), read depth (<10) alternate allele frequency (<20%), and presence in <0.1% of cells. The single cell MRD computational demultiplexing and single cell MRD protein analysis are described below in further detail.

3.1.5 scMRD Computational Demultiplexing

Deconvolution of multiplexed scMRD runs was reliant on the presence of germline SNPs. Suspected SNPs were verified via referencing the Ensembl SNP database through the BioMart R package and were tallied for non-missing genotyping information within the filtered NGT matrix. The top 10-20 SNPs with the lowest percentage of missing genotypes were selected for downstream analysis. K-means clustering was performed on SNP allele frequencies in a subset of cells with complete SNP genotypes, where the number of clusters for partitioning was set equal to the number of unique patient samples in a given multiplex. Doublet identification and exclusion was conducted by first evenly sampling cells from all clusters to form a pool of cells with equal representation of each cluster. Artificial doublets were then generated via sampling the cell pool two cells at random and averaging the SNP profiles until the proportion of artificial doublets approached 5-10% of the total number of cells in the dataset. Doublets were then merged with real cells and re-clustered to produce real and artificial cluster centers. The Euclidean distance was then measured between each real cell and 1) it's respective cluster, 2) the artificial cluster center. The distribution of distances between 90-95% of cells to their respective cluster centers was used as a cutoff to exclude cells which were within this distance to the artificial cluster center. This process was repeated 10 times, with random replacement of NA values with allele frequencies of 0, 50, or 100, and cells were excluded if their distance was within the doublet gate in all replicates. After removing doublets and low-quality cells with high similarity to artificial doublets, the most common SNP profile was tallied for each cluster. To classify additional cells, a Hamming distance was calculated between all cells and each SNP profile, without penalizing SNPs with missing genotypes. Cells were assigned to clusters based on matching 80% of the SNP profile and being the maximum Hamming distance from every other cluster. For some multiplexed runs, slightly less stringent filters were applied to reduce the Hamming distance between clusters. After cell classification, each cluster was queried for pathogenic mutations detected by bulk NGS at the diagnosis, remission, and relapse (if applicable) timepoints, and the cell number per cluster was tallied.

### 3.1.6 Single Cell Protein Analysis

For each demultiplexed sample, single cell protein data was extracted from H5 files as raw counts. Each demultiplexed patient sample was analyzed independently for clonality of mutations and clone-specific immunophenotype. For samples with detected mutations, the protein count matrices were filtered for cells classified into high-confidence clones (>3 cells) and were used for subsequent aggregate analysis. Protein counts for each run were merged and converted to a Seurat object using the Seurat R package. The protein data was log-normalized, scaled, and centered on a by-run basis. Clone and mutation information was supplied as metadata and used for downstream aggregate analysis using functions within Seurat.

### 3.1.7 Limit of Detection Study Analysis

For each multiplexed AML spike-in run, the numerical genotype matrix (NGT) was extracted from each respective H5 file in R. Each of the three AMLs harbored >1 pathogenic mutation, except for patient 3 which contained a single IDH2.R140Q mutation. To increase the confidence in accurate cell calling, two additional heterozygous germline SNPs (CHEK2.T387I and TET2.P1723S) were identified as private to patient 3. The curated list of known variants included mutations/SNPs present in patient 1 (NRAS.G12D, RUNX1.P247fs), patient 2 (DNMT3A.F751fs, JAK2.V617F, IDH2.R140Q), and patient 3 (IDH2.R140Q, CHEK2.T387I, TET2.P1723S). After filtering, all cells were queried for variants included in this list. Cells harboring the expected variants were then filtered based on the requirement that real cells must contain at least two pathogenic mutations (patients 1 and 2), or one pathogenic mutation and two SNPs (patient 3). Limiting dilution analysis was conducted using the Extreme Limiting Dilution Analysis software, where the AML spike-in cell number was treated as ‘Dose’, and the number of replicates in which the leukemic fraction was detected was treated as ‘Response’. Output of the analysis provided an estimated sensitivity with an associated confidence interval.

### 3.1.8 Plotting and Graphical Representation

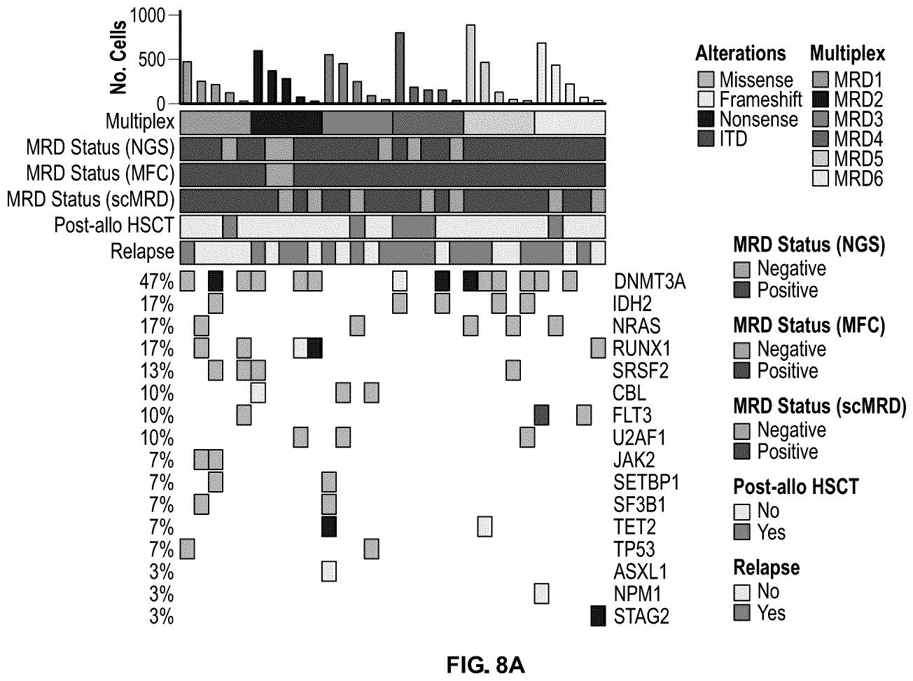

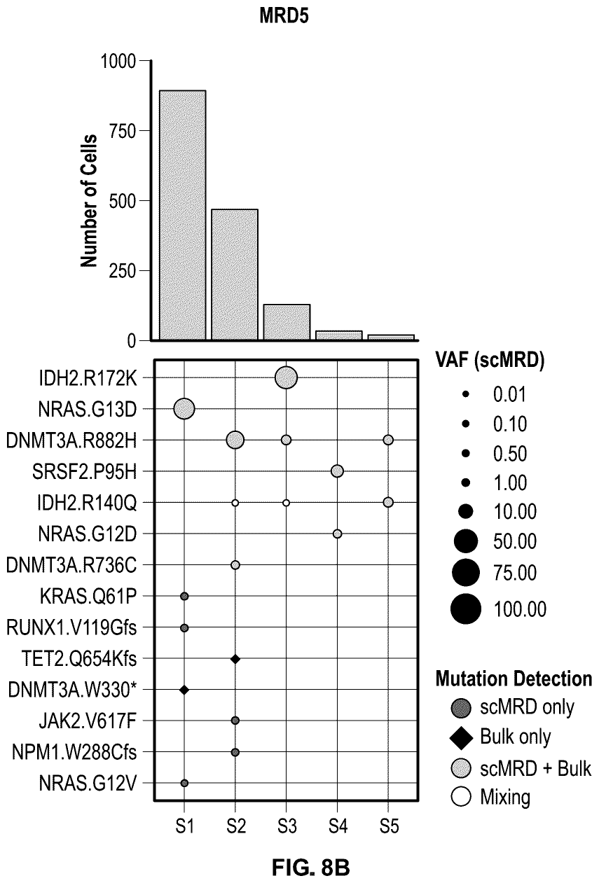

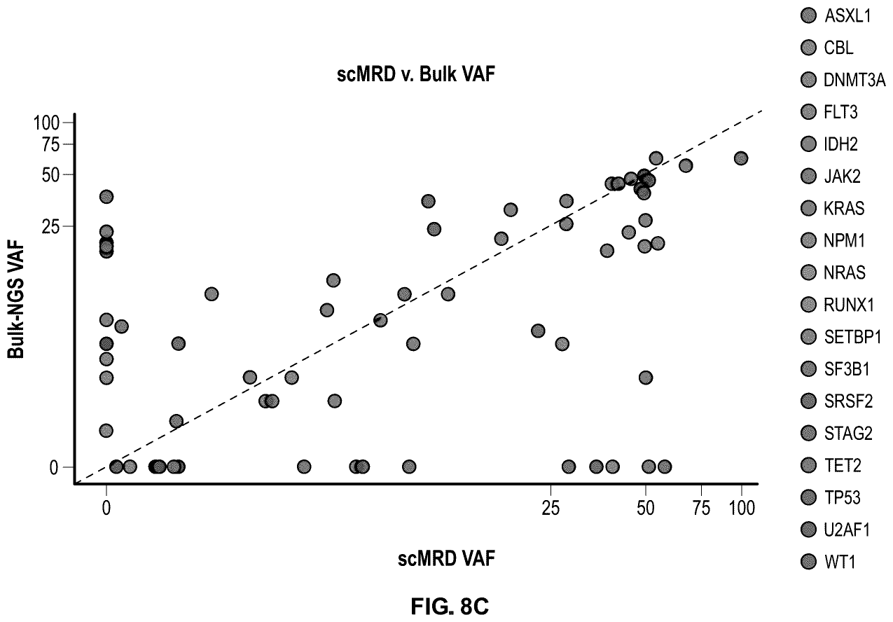

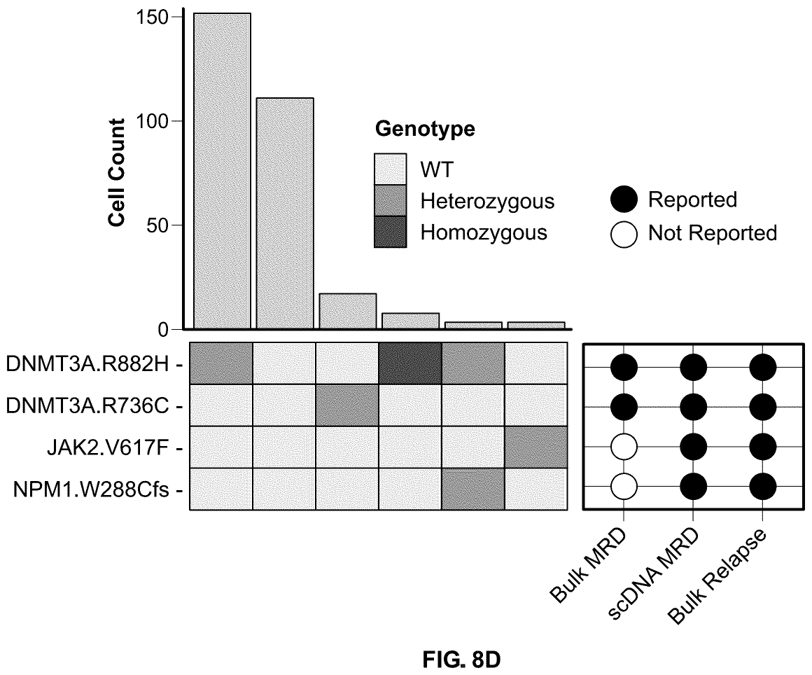

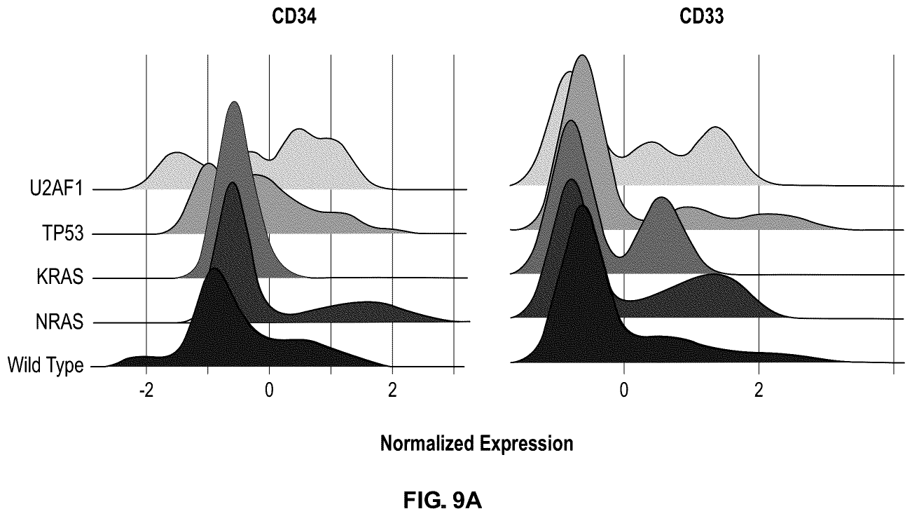

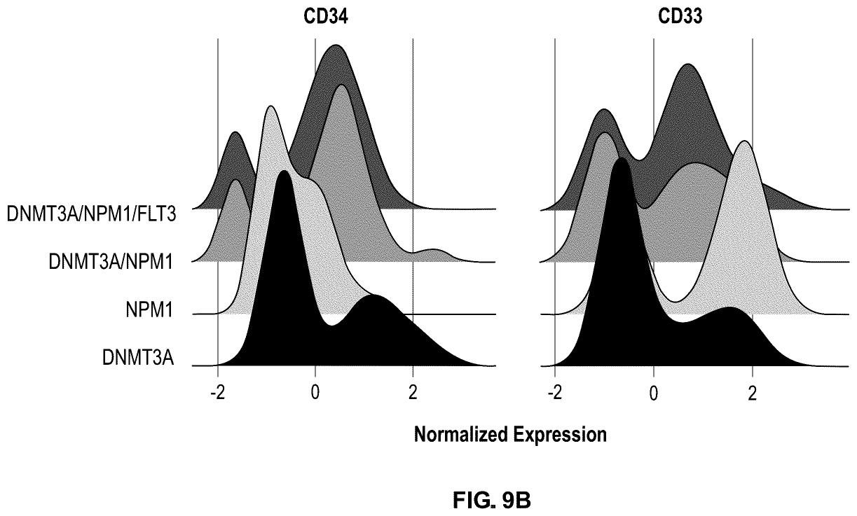

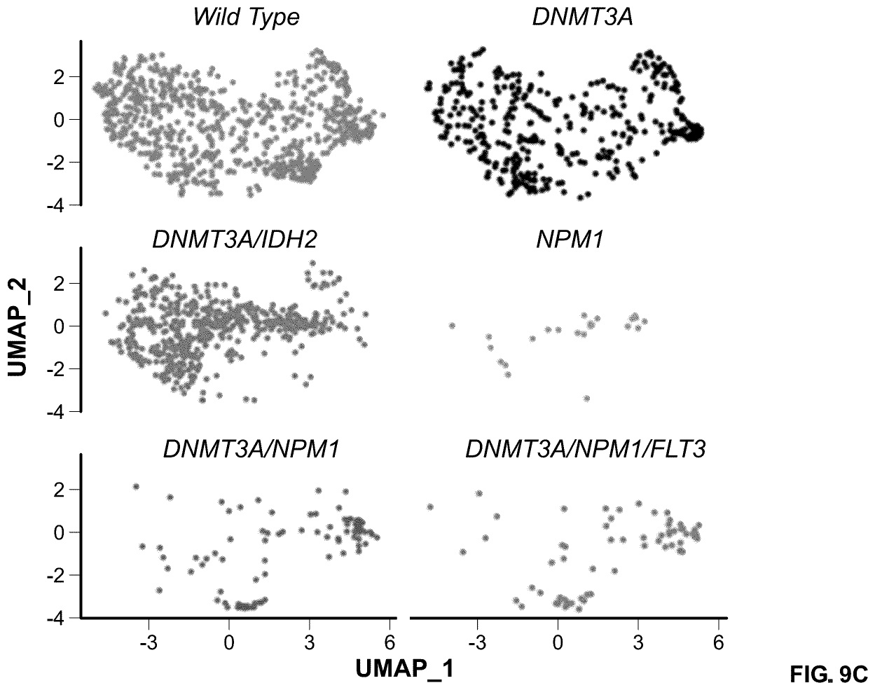

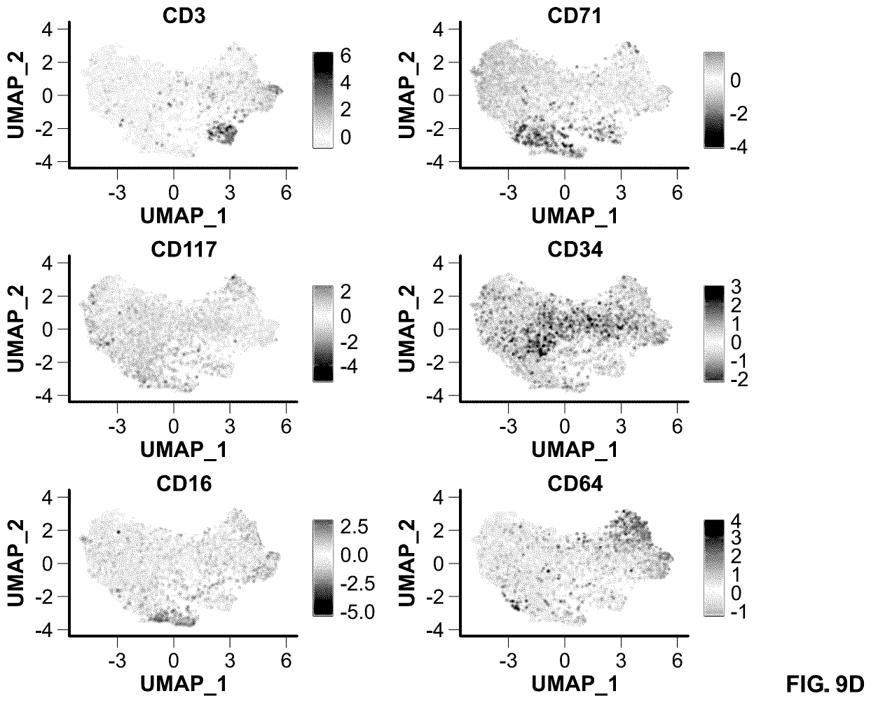

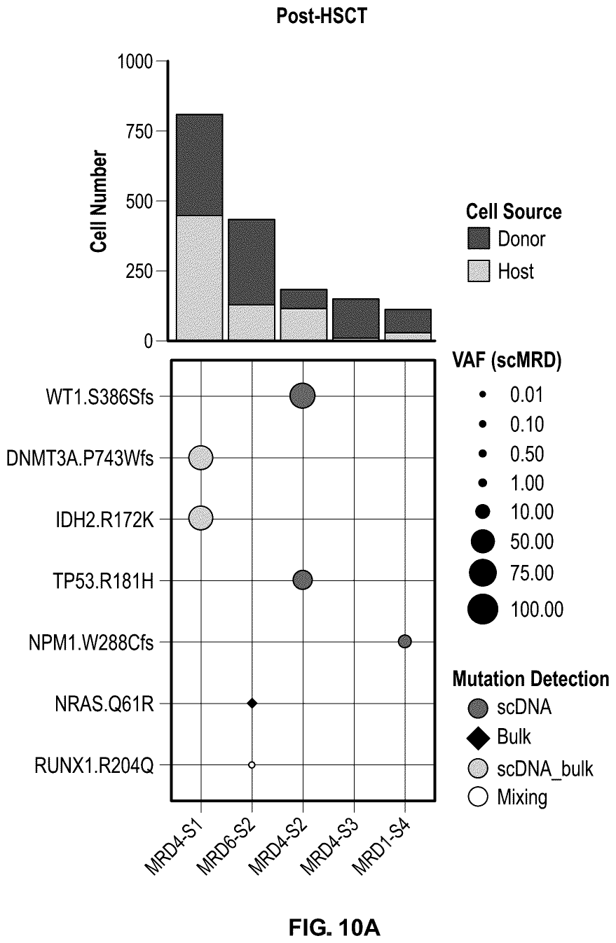

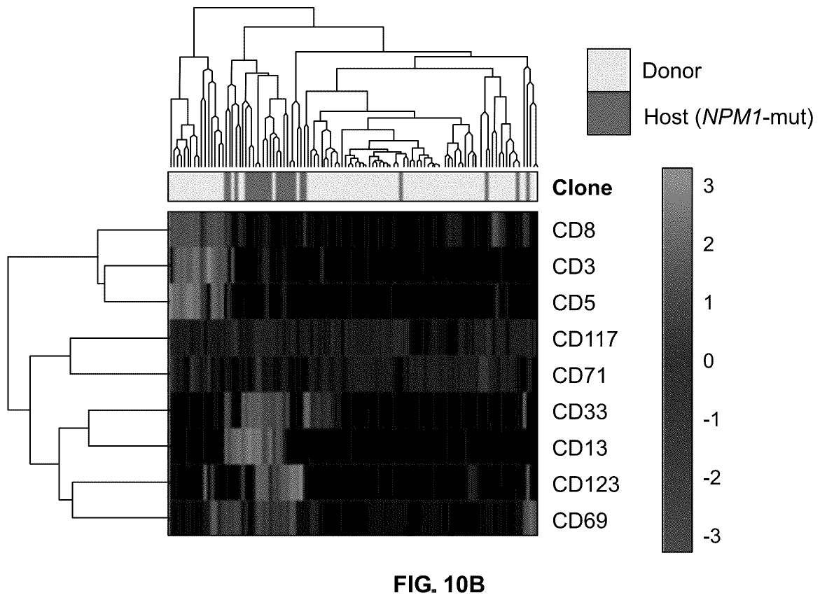

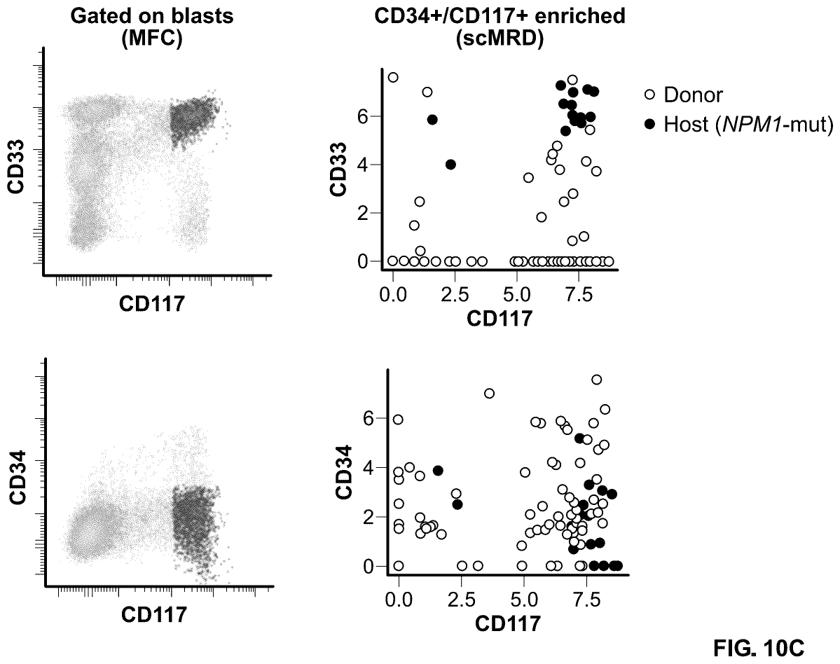

All bar plots and scatter plots were generated using the ggplot2 package in R. For example, the OncoPrint shown in FIG. 8A was produced using the Complex Heatmap package in R. All heatmaps were generated using the pheatmap R package. The UMAP plots, density plots, and violin plots, in FIG. 9 and Supplementary FIG. 10 were generated using the Seurat R package. The radar plot displayed in Supplementary FIG. 10 was produced with the fmsb package in R.

### 3.2 Results

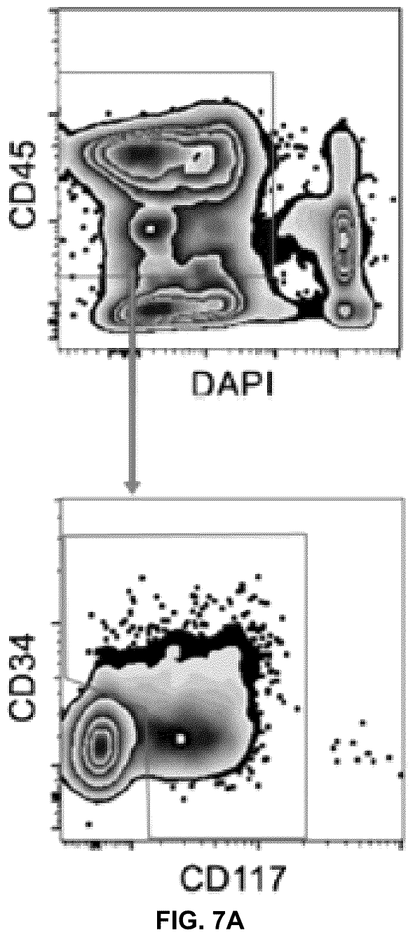

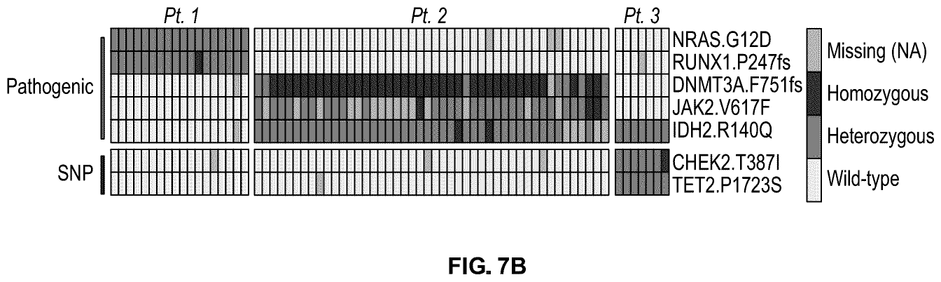

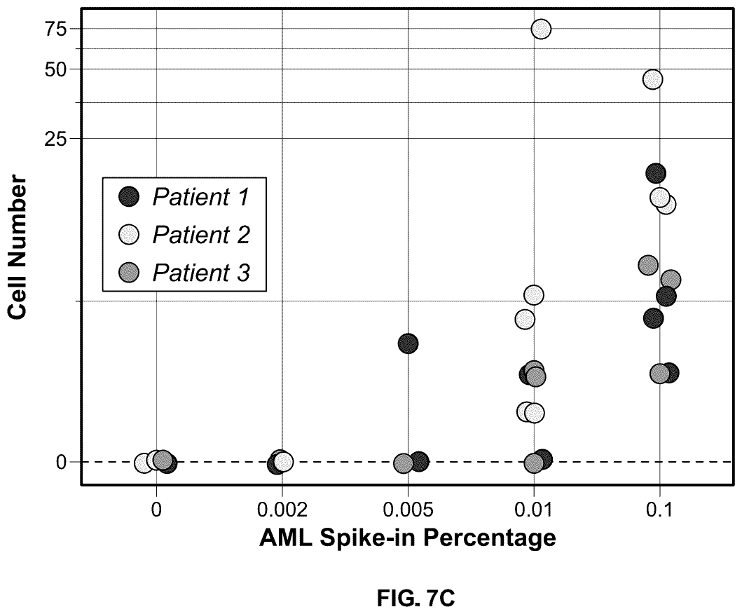

FIGS. 7A-C illustrate limit of mutation detection with the scMRD assay. FIG. 7A illustrates schematic of gating strategy for flow cytometric enrichment of live CD34+ and/or CD117+ cells that were sorted. For clinical samples, the abnormal blasts were positive for CD34 and/or CD117. FIG. 7B illustrates representative heatmap showing mutation calling of spiked-in AML blasts in a limit of detection experiment testing a sensitivity of 0.1%. FIG. 7C illustrates a summary of mutation detection at various sensitivity levels. This plot represents two independent experiments.

FIGS. 8A-8D illustrate mutation and relapse associated clone identified by scMRD assay. FIG. 8A illustrates Oncoprint showing concordance of MRD detection by bulk NGS assay, scMRD assay and MFC. Bar plot (top) represents the number of cells recovered after computational demultiplexing. Mutations represent those that were detected by bulk NGS at the remission timepoint and are covered by the custom scDNA panel. Post-allo HSCT represents the time of MRD assessment. Relapse represents outcomes after MRD assessment. FIG. 8B illustrates a representative deconvolution plot of one multiplexed scMRD run. FIG. 8C illustrates comparison of mutations detected by bulk NGS vs scMRD. FIG. 8D illustrates Clonograph of a patient (MRD5-S2) illustrating scMRD-specific detection of NPM1 and JAK2 mutations that were present at late relapse.

FIGS. 9A-9D illustrate clone-and mutation-specific immunophenotype. FIG. 9A illustrates clone specific immunophenotype. FIG. 9B illustrates differential surface marker expression between CH/preleukemic vs leukemic clones. FIGS. 9C and 9D illustrate UMAP analysis of immunophenotypes of CH/preleukemic vs leukemic clones. Data are lognormalized, centered, and scaled on a by-run basis.

FIGS. 10A-10C illustrate scDNA+protein analysis that enables simultaneous identification of donor cells and MRD. FIG. 10A illustrates aggregated deconvolution plot showing mutations detected and host-donor chimerism of post-allogeneic HSCT samples included in the study. MRD4-S3 had an HDACI P243L mutation not covered by the scMRD panel. FIG. 10B illustrates Heatmap analysis of differential surface maker expression between donor and host cells in MRD1-S4. FIG. 10C illustrates concordance of immunophenotype of MRD cells between MFC and scMRD in MRD1-S4.

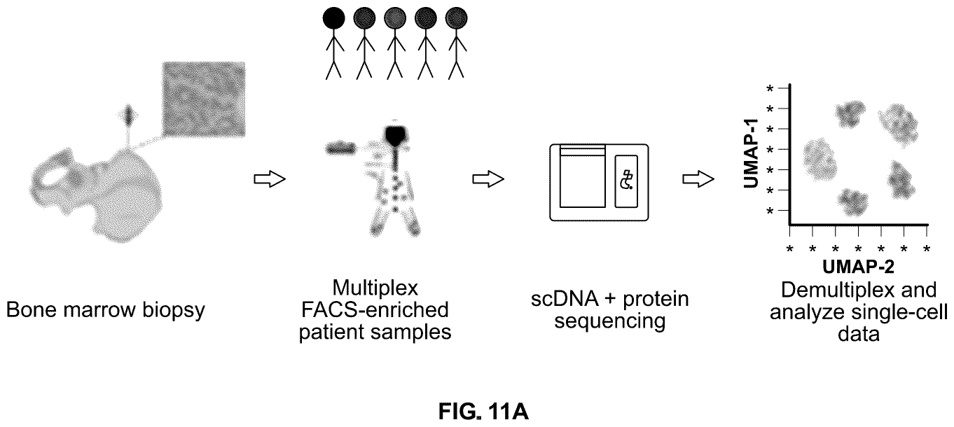

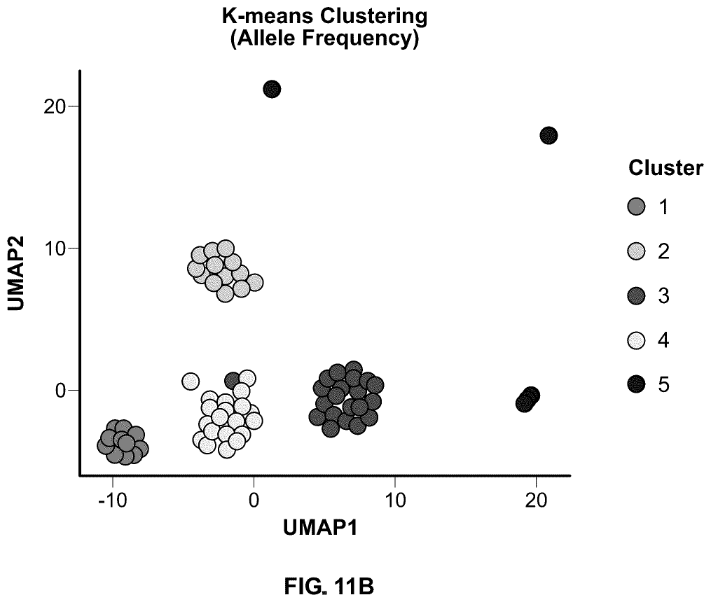

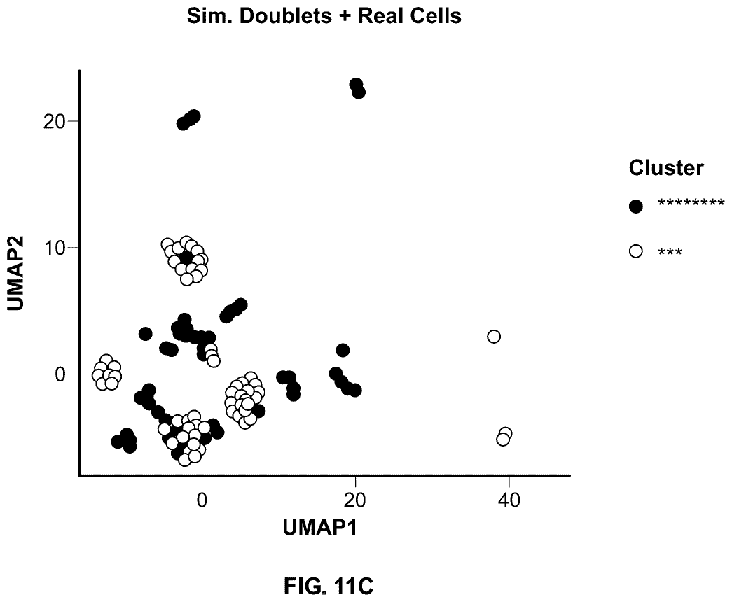

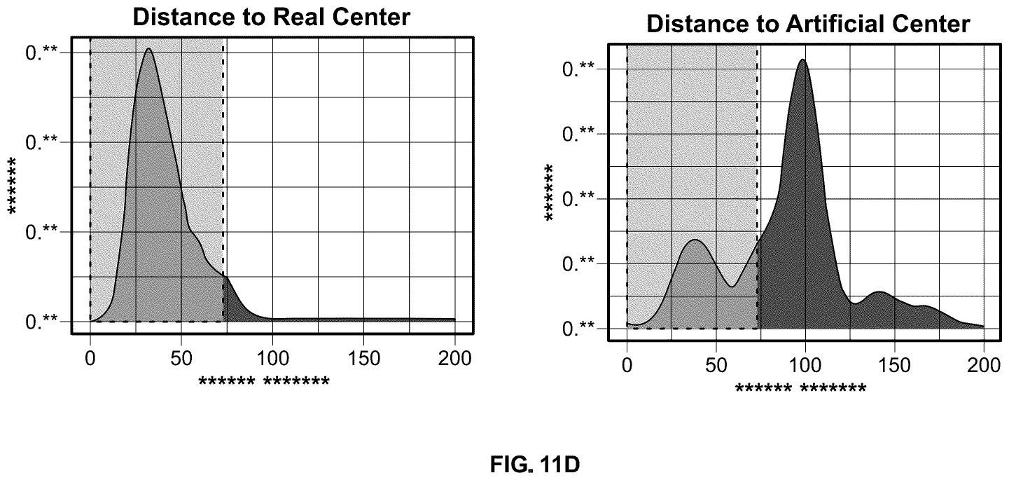

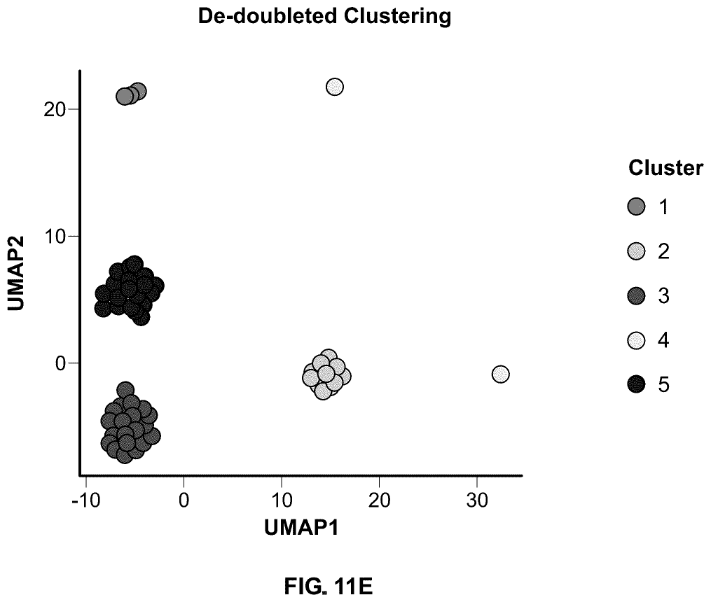

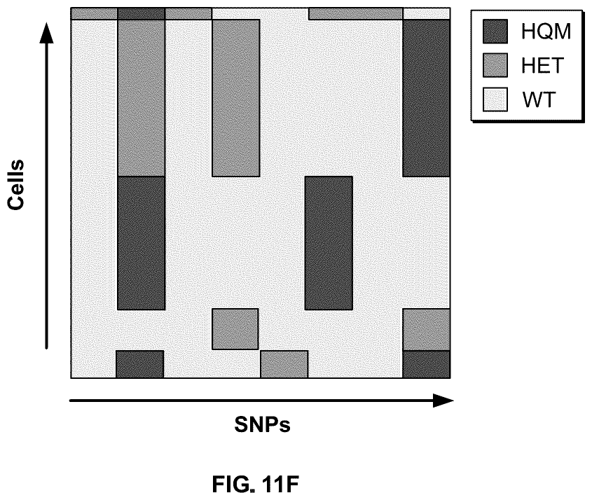

FIGS. 11A-11F illustrate a workflow and computational demultiplexing of scMRD data. FIG. 11A illustrates schema of scMRD workflow (e.g., generated via BioRender). The panels in FIGS. 11B-11F show representative examples of the computational pipeline output. More specifically, FIG. 11B illustrates K-means clustering and UMAP analysis of SNP allele frequencies before doublet exclusion. FIG. 11C illustrates UMAP plot showing the results of clustering real cells (left) with artificial doublets (right). FIG. 11D illustrates distribution of Euclidean distances from real cells to their respective cluster centers (left) and to the artificial cluster center (right). FIG. 11E illustrates K-means clustering and UMAP analysis of SNP allele frequencies after doublet exclusion. FIG. 11F illustrates heatmap showing private SNP genotypes in singlet clusters.

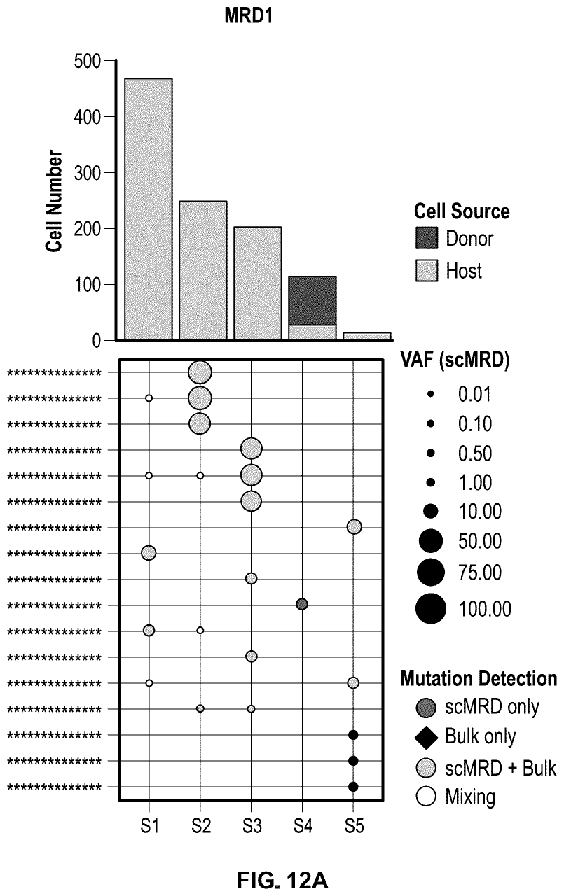

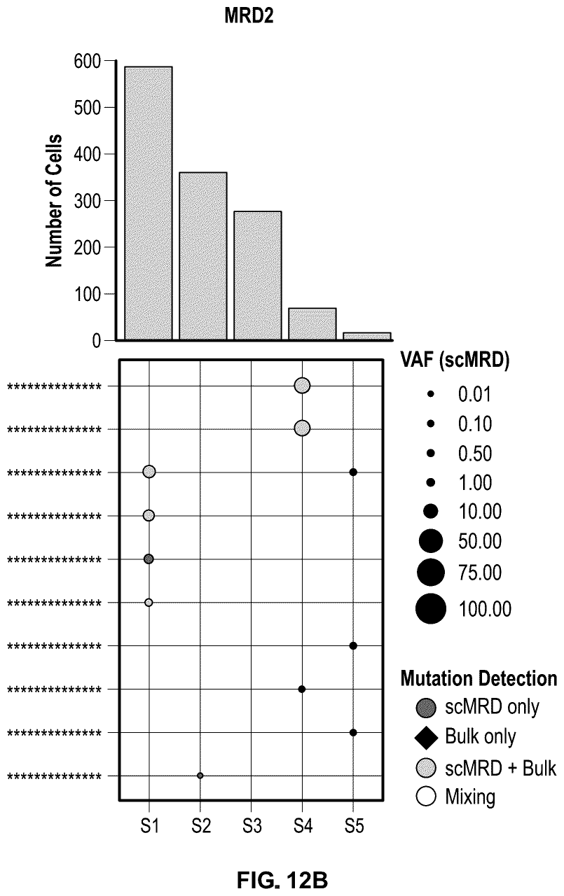

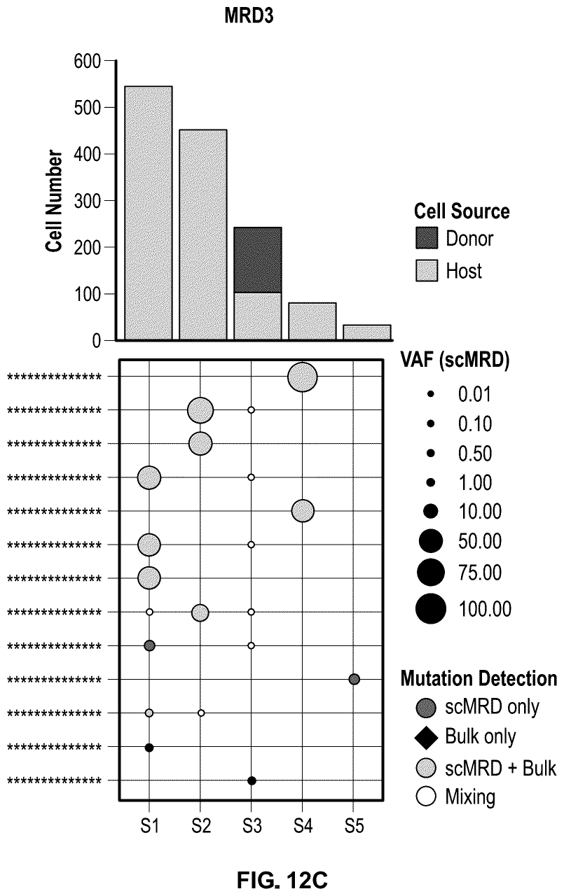

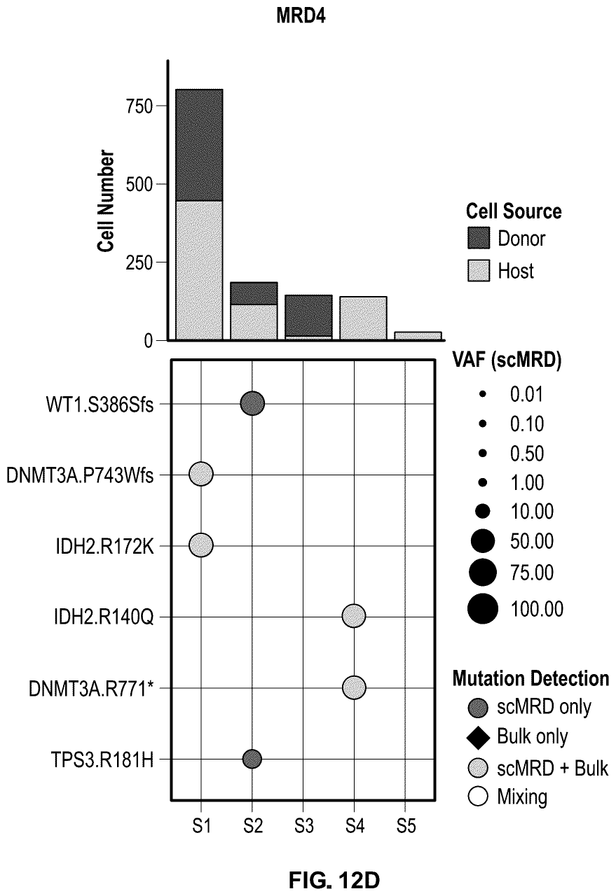

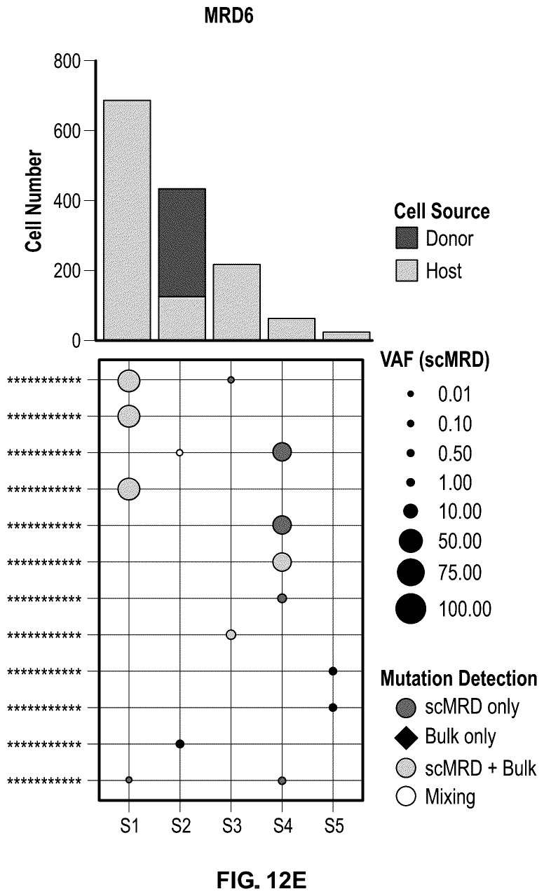

FIGS. 12A-12E illustrate deconvolution plots for scMRD runs. More specifically, FIGS. 12A-12E illustrate recovered cell number per sample (top) and VAF of mutations detected by scMRD, bulk NGS, or both assays (bottom). Mixing represents mutations found in ≤2 cells that were likely misclassified by the demultiplexing pipeline.

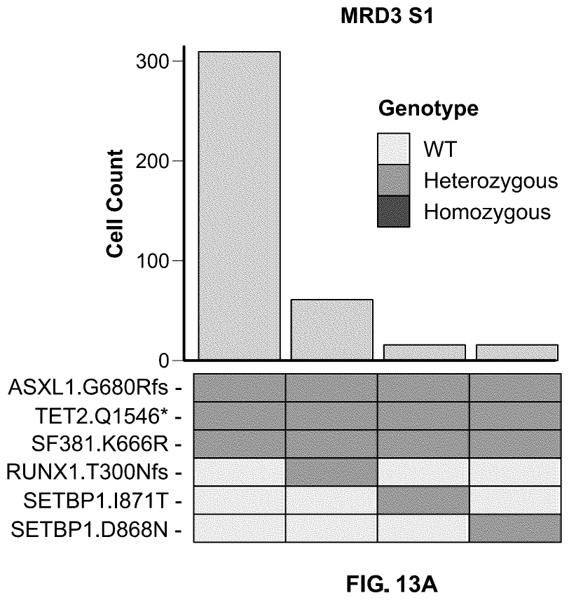

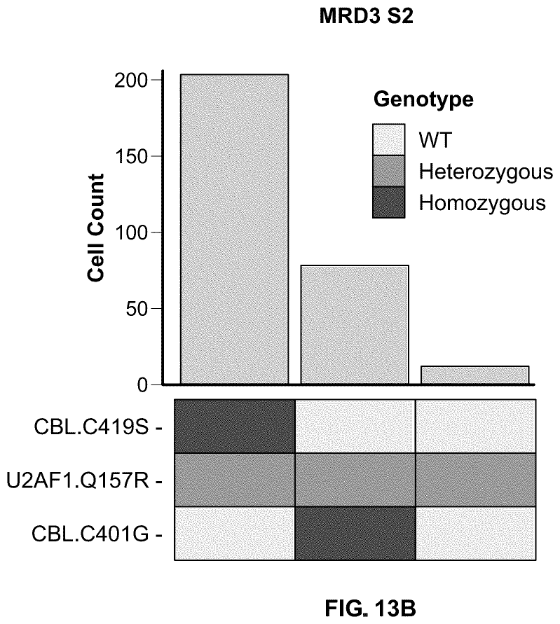

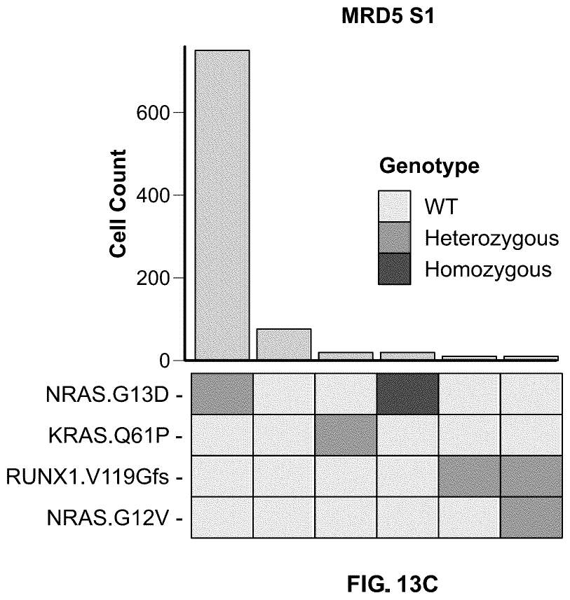

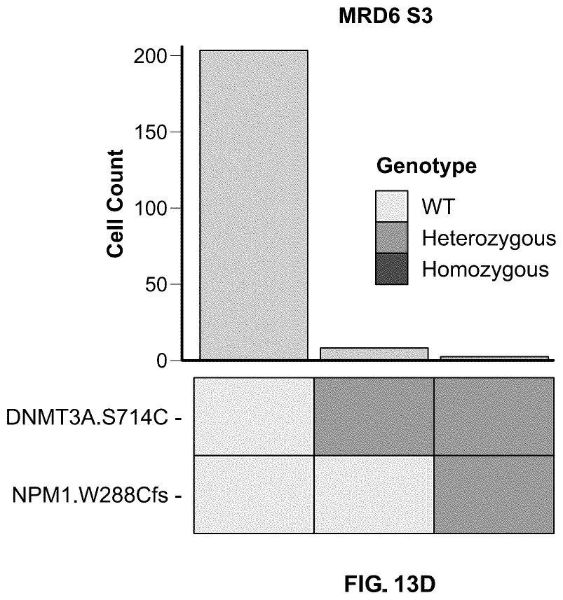

FIG. 13A-13D illustrate representative clonographs of MRD samples, in which columns represent individual clones identified in each sample, with cell count (top, bar plot) and zygosity of mutations present (bottom, heatmap).

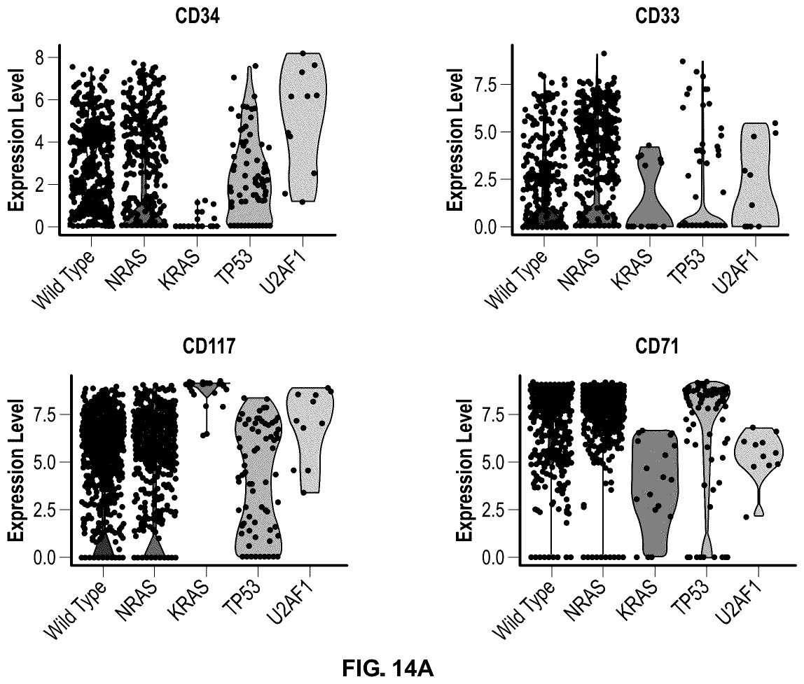

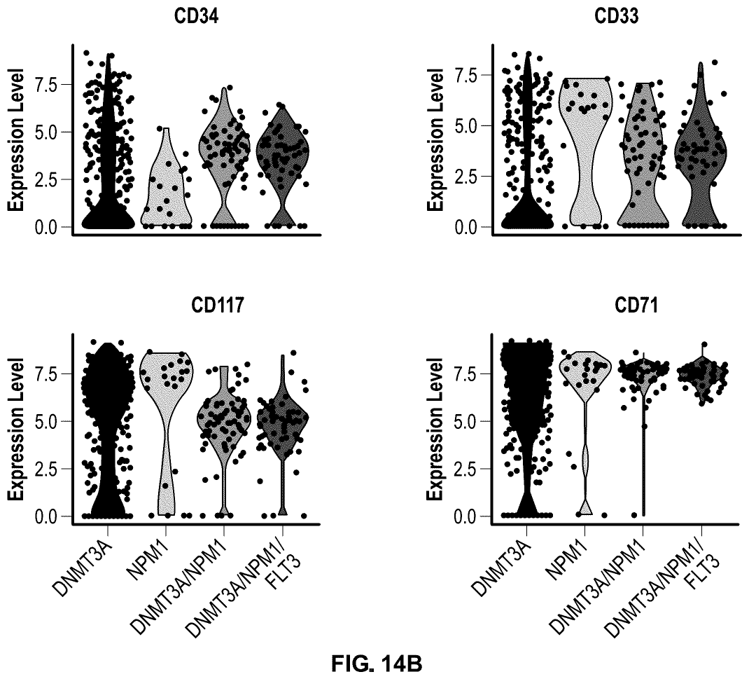

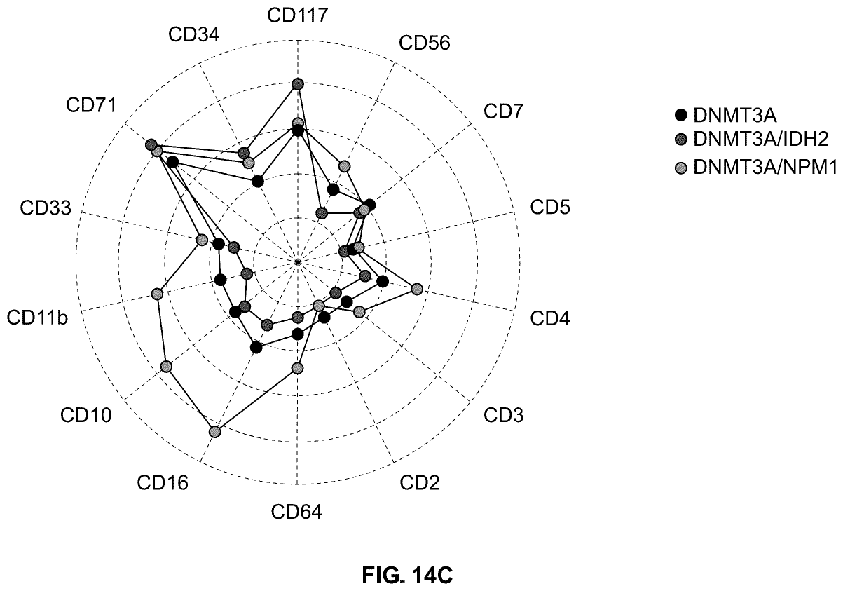

FIGS. 14A-14C illustrates analysis of protein sequencing data of MRD clones. FIG. 14A illustrates violin plots showing log-normalized differential surface marker expression of various MRD clones. FIG. 14B illustrates violin plots showing log-normalized differential surface marker expression of CH/preleukemic (DNMT3A) vs leukemic (NPM1, DNMT3A/NPM1, DNMT3A/NPM1/FLT3ITD) clones. FIG. 14C illustrates radar plot showing differential surface marker expression of CH/preleukemic (DNMT3A) vs leukemic (DNMT3A/NPM1, DNMT3A/IDH2) clones. Each marker is scaled relative to the maximum and minimum expression values for all cells with DNMT3A, DNMT3A/NPM1, or DNMT3A/IDH2 mutations.

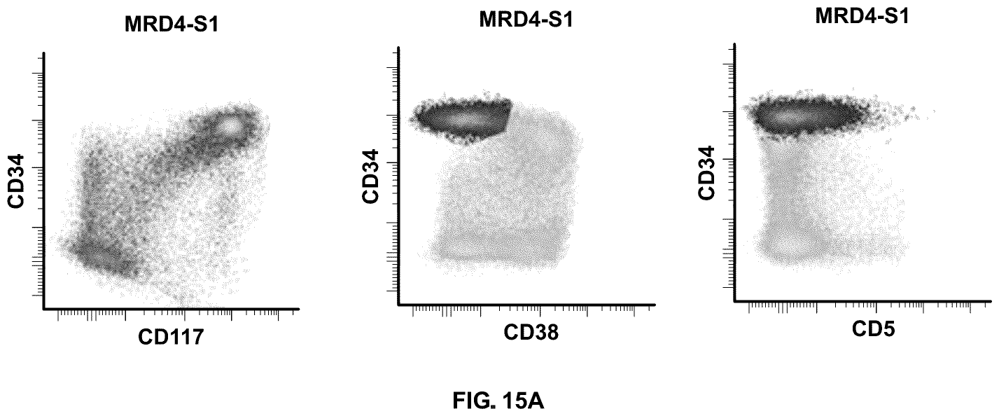

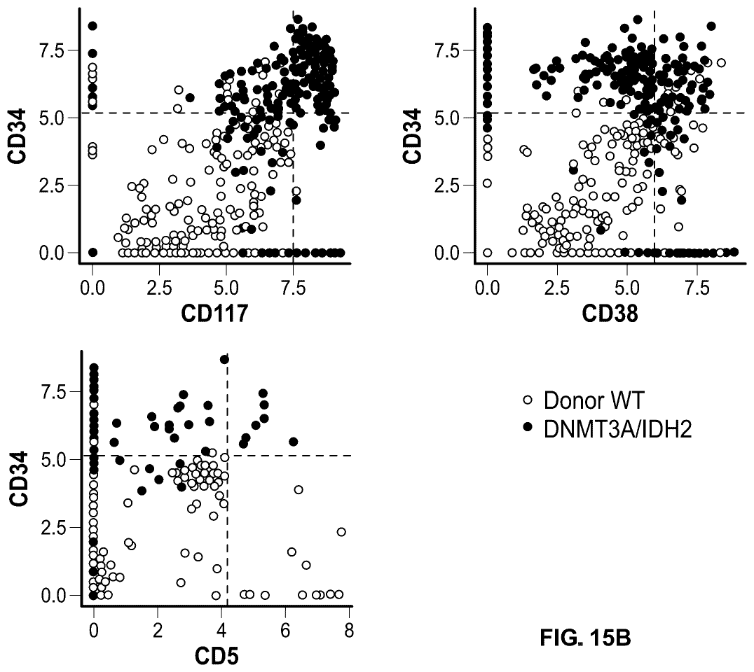

FIGS. 15A and 15B illustrates concordance of immunophenotype between MFC and scMRD assay from a representative patient (MRD4-S1). FIG. 15A illustrates flow plots showing abnormal expression of bright CD117, dim to negative CD38 and partial CD5 on CD34 positive myeloblasts. FIG. 15B illustrates scMRD data shows similar immunophenotype.

As shown in FIG. 7A, flow cytometry assisted cell sorting (FACS) were utilized to enrich viable CD34+ and/or CD117+ progenitors, prior to loading cells onto the Mission Bio Tapestri single cell sequencing platform (FIG. 7A). The custom single cell DNA panel contained 109 amplicons covering 31 genes known to be involved in hematologic malignancies. To increase assay throughput, samples from different patients were multiplexed into each integrated single cell DNA+protein run. The results of the multiplexed runs were then computationally deconvoluted and used for both single-sample and aggregated analyses.

To evaluate the sensitivity of the single cell MRD assay, a limiting dilution study was performed by mixing AML blasts from 3 genetically distinct AML samples harboring clonal mutations with 10 million normal bone marrow mononuclear cells to test different sensitivity thresholds (10,000 cells 0.1%, 1,000 cells 0.01%, 500 cells 0.005% and 200 cells 0.002%). Mutations were as follows: Patient 1: NRAS p.G12D/RUNX1 p.P247fs, Patient 2: JAK2 p.V617F/IDH2 p.R140Q/DNMT3A p.F751fs, Patient 3: IDH2 p.R140Q).

As shown in FIG. 7B, FACS-enriched CD34+ and/or CD117+ cells were multiplexed, subjected to the Tapestri v2 microfluidics platform, and sequenced. Expected pathologic mutations were identified in all 11 replicates at a sensitivity of 0.1% (FIG. 7B).

As shown in FIG. 7C, mutations were also identified in 8/10 and ⅓ replicates at a threshold of 0.01% and 0.005%, respectively, and mutations were not identified in blank controls (0/9 replicates) or when present at 0.002% (0/3 replicates) (FIG. 7C). Limiting dilution analysis estimated a sensitivity of 0.0077% (95% CI [0.004%-0.0153%]). These data demonstrate the high sensitivity and specificity of mutation detection using the single cell MRD assay.

Further, the single cell MRD assay was applied to 30 cryopreserved post-induction chemotherapy MRD samples obtained from 29 AML patients (median age 71 years old, 15 male and 14 female).

As shown in FIG. 8A, MRD was scored as negative in 2 samples by MFC and in 6 samples by bulk NGS. The median cell number of these samples was 2.6 million (ranging from 0.6-14.1 million) with a viability range of 27-55%.

Further, as shown in FIG. 11A, FACS-enriched CD34+ and/or CD117+ viable cells were multiplexed with up to 5 unique patient samples per run and processed via the Tapestri platform (50-100 thousand cells per run, median 65 thousand).

Given that different samples from multiple patients were included in each single cell MRD run, a computational approach was developed to deconvolute different individuals in each sequencing run at the single cell level, as shown in FIG. 11. More specifically, downstream demultiplexing of single cell MRD sequencing data used germline single nucleotide polymorphisms (SNPs) covered by the custom single cell DNA panel. The dataset was filtered based on genotyping call rate to include cells with complete genotyping information for the top 10-20 SNPs in each run and performed K-means clustering on cells with non-missing SNP allele frequencies. To identify and remove doublets within each single cell MRD run, the method was implemented to simulate artificial doublet SNP profiles and exclude putative real doublets based on similarity to an artificial cluster center. The dataset was first randomly sampled to produce a pool of cells with even representation of each cluster, followed by sampling two cells in the cell pool at a time, averaging their SNP allele frequency profiles, and re-clustering the artificial doublets with real cells. Then, a Euclidean distance metric was applied to assess the similarity between the SNP profiles of real cells and artificial cluster centers. After doublet detection and exclusion, additional cells in the dataset was then classified according to their germline SNP profile. To achieve this, a Hamming distance between each cell and the most common SNP profile was calculated for each cluster. Cells were assigned to clusters based on the SNP profile matching at least 80% of one cluster while being the maximum Hamming distance from every other cluster.

This approach enabled deconvoluting multiplexed single cell MRD runs and assign sequenced cells to the specific patient from which they were derived without the need to leverage patient-specific somatic mutation information. For example, using this approach, an average of 1,333 sorted cells per run (1,053−1,544 cells, SD=163.4) was classified.

As shown in FIGS. 8B, 11, and 12, demultiplexing enabled assignment of hotspot mutations (e.g., DNMT3A p.R882H) present in multiple samples within the same multiplex.

Overall, the results for MRD status and mutation presence were concordant between bulk NGS and single cell MRD in 22/30 (73%) samples and for 46/77 (60%) mutations, respectively (FIG. 8A). Mean variant allele frequencies (VAF) of mutations detected by both single cell MRD and bulk NGS trended towards higher allele burden by single cell MRD (p=0.064, paired Wilcoxon signed rank test) (FIG. 8C). Among the 31 discordant mutations covered by both single cell MRD and bulk NGS panels, single cell MRD identified 17 mutations that were missed/unreported by bulk NGS, including RUNX1 (n=5), NPM1 (n=3), KRAS (n=2), IDH2 (n=2), WT1 (n=2), JAK2, TP53 and SRSF2 mutations (FIG. 12), 14 (82.4%) of which were associated with and present at relapse. Conversely, there were 14 mutations that were detected by bulk NGS but missed by single cell MRD, including DNMT3A (n=4), RUNX1 (n=3), NRAS (n=2), STAG2, TET2, SETBP1, SRSF2 and FLT3TKD mutations. Interestingly, only 6/14 (42.8% vs 82.4%, p=0.03, Fisher exact test) were present at relapse. There were 4 MRD samples with 7 mutations (2 RUNX1, 2 DNMT3A, 2 NRAS, and STAG2) not detected by single cell MRD. Although these samples had slightly lower viable and recovered cell numbers compared to others (median: 1.5 million vs 2.6 million [p=0.08], 64 vs 175 [p=0.1], respectively, Mann-Whitney test), the cause may be multifactorial including that the presence of specific mutations (i.e. NRAS) may reside in mature/differentiated compartments not sampled with our single cell MRD assay.

Further, the ability of single cell MRD assay profiling was assessed to differentiate MRD based on clonal architecture, including discrimination between single mutant CH/pre-leukemic and leukemic clones. We found that single cell MRD readily deconvolved CH/pre-leukemic vs. leukemic clonal architecture in demultiplexed samples (FIGS. 8D and 13). In sample MRD5-S2, bulk NGS detected DNMT3A p.R882H, DNMT3A p.R736C, and TET2 p.Q654Kfs (not covered by single cell MRD panel) mutations at the remission timepoint, while single cell MRD detected both DNMT3A mutations in distinct clones, with one subclone harboring DNMT3A p.R882H/NPM1 p.W288Cfs (NPM1 sc VAF=1.22%) co-occuring mutations and another with a JAK2 p. V617F (JAK2 scVAF=0.23%) mutation. Importantly, bulk NGS at the time of subsequent relapse revealed the presence of both the NPM1 p. W288Cfs (VAF=5%) and JAK2 p.V617F (VAF=2%) mutations (FIG. 8D). These data demonstrate that single cell MRD enables resolution of residual pre-leukemic clones (i.e. DNMT3A alone) and leukemic clones (co-mutant DNMT3A/NPM1) that persist at relapse.

Integration of single cell MRD immunophenotypic analysis enabled identification of mutation and clonespecific expression of key cell surface proteins (FIGS. 9A, 9B, 14A, and 14B). Compared to wild type clones, single mutant clones displayed differential expression of CD34, such as U2AF1 (log2FC=3.5, P<0.002) and KRAS (log2FC=−5.72, P<0.02). Interestingly, NRAS-mutant clones had a marked increase in expression of CD33 (log2FC=1.47, P<1.19×10−6) but not CD34 (log2FC=0.20, P<6.16×10−10) consistent with a previous study.

Differential immunophenotypic states were identified when comparing CH/pre-leukemic and leukemic clones within and between patients (FIGS. 9B-9D, 14B, and 14C).

Compared to compound mutant DNMT3A/NPM1 (with or without FLT3) clones, single mutant NPM1 clones showed reduced CD34 (log2FC=−2.75, P<0.0049) and increased CD117 (log2FC=2.68, P<0.043) surface expression. In addition, NPM1 clones displayed higher CD33 expression (log2FC=1.03, P<0.04) compared to DNMT3A clones. This distinct immunophenotype is well described in patients with NPM1-mutated AML14. Highly similar immunophenotypes were observed between wild type and DNMT3A-mutant cells, suggesting CH clones (at least with DNMT3A mutation) may not have overtly aberrant surface protein expression (FIGS. 9C-9D). By contrast, co-mutant DNMT3A/IDH2 or DNMT3A/NPM1 cells showed consistently aberrant immunophenotypes, with DNMT3A/IDH2 cells characterized by increased CD71 expression (log2FC=0.388, P<8.7×10−8, vs. DNMT3A) (FIGS. 9C-9D and FIGS. 14B-14C), indicative of erythroid biased differentiation consistent with previous reports. Concordant with the findings in FIG. 9B, the overall patterns of surface protein expression were different between NPM1-mutated and DNMT3A/NPM1 co-mutated cells with the latter more highly expressing CD34 and granulocytic/monocytic markers such as CD16 (log2FC=3.37,P<1.2×10−9, Percent Expressin =98.9%) and CD64 (log2FC=3.05, P<7.7×10−4, Percent Expressing=75.9%) (FIG. 9D). These data highlight that integrated genomic/immunophenotypic analysis at the MRD time point can distinguish between CH/preleukemic and leukemic clones that portend a substantively higher likelihood of relapse. Within our cohort, 5 samples were obtained post-allogeneic hematopoietic stem cell transplant (HSCT) and represented an admixture of donor and recipient cells.

Germline SNP-based deconvolution identified distinct non-mutant clusters consistent with donor origin, which were confirmed as donor samples by matching the SNP profile of paired pretransplant samples. Donor-host pairs were recovered for all 5 samples (FIG. 10A). Chimerism was calculated based on recovered host vs donor cells and correlated with the results by bulk short tandem repeat genotyping (STR) on unsorted BM samples. The levels of host cells detected by single cell MRD assay were significantly higher than those by STR testing in 4 relapsed patients (median of difference 30%, p=0.04, Wilcoxon matched pairs signed rank test), suggesting that host cells show enrichment in immature compartments and represent an early indicator of relapse. Integrated immunophenotypic analysis of post-allogeneic HSCT samples showed distinct cell surface protein expression between donor and host cells. Analysis of sample MRD1-S4 revealed clear separation of donor and NPM1-mutant host cells, with the former containing a subset of spiked-in CD3+CD8+ T-cells, and the latter displaying aberrant increased expression of CD33 (log2FC=2.54, P<1.32×10−4), CD13 (log2FC=3.9, P<1.06×10−5), and CD123 (log2FC=4.08, P<0.011) (FIG. 10B), of which CD123 is a well described leukemic stem cell marker. In addition, expression of the T-cell activation marker, CD69 was observed in a subset of donor and host T cells but also unexpectedly in host leukemic cells, consistent with previous studies showing that CD69 may be expressed in leukemic stem cells and thus may represent a surface marker for MRD detection. Further, abnormal immunophenotype of these host leukemic blasts identified by MFC was also detectable by single cell MRD, with elevated co-expression of CD33 and CD117 on NPM1-mutant cells and characteristically low levels of CD34 (FIG. 10C).

According to the discussion above, this example illustrates the feasibility of single cell genotypic and immunophenotypic profiling at the remission timepoint to enumerate and delineate MRD through blast enrichment and single cell DNA+protein technology. The data demonstrates that single cell MRD profiling readily resolves clonal architecture and can distinguish between single mutant CH/pre-leukemic vs. leukemic clones with multiple co-occurring mutations. The integration of mutation and immunophenotypic information further enhances MRD detection by identifying genotype-specific protein expression patterns. This can be potentially utilized to isolate relevant clones for studying MRD biology and therapeutic vulnerabilities. Given the increased use of molecular/cell surface-targeting therapeutic modalities for AML patients, assessing expression of surface markers in relevant MRD clones with defined mutational repertoires may provide further guidance for treatment.

Thus, the single cell MRD assay as described in enables sensitive MRD detection, as well as achieves resolution to characterize the clonal architecture of pre-leukemic/leukemic cells that persist after therapy, which may increase the specificity of MRD results.

### Example 4

### Example Results Using Methods and Systems for Analyzing MRD-Relevant Cells

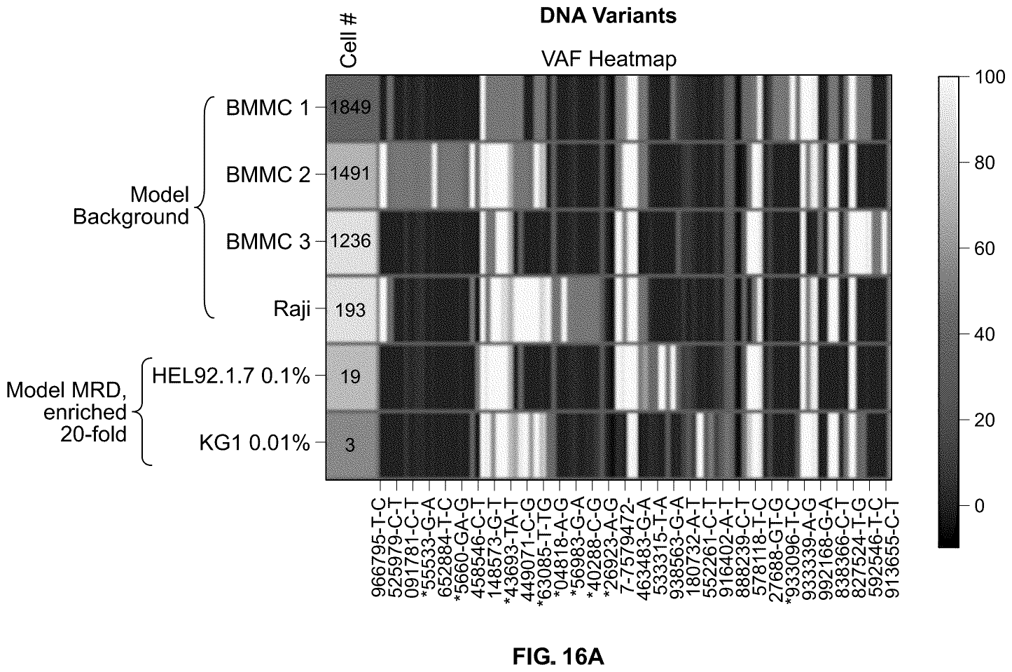

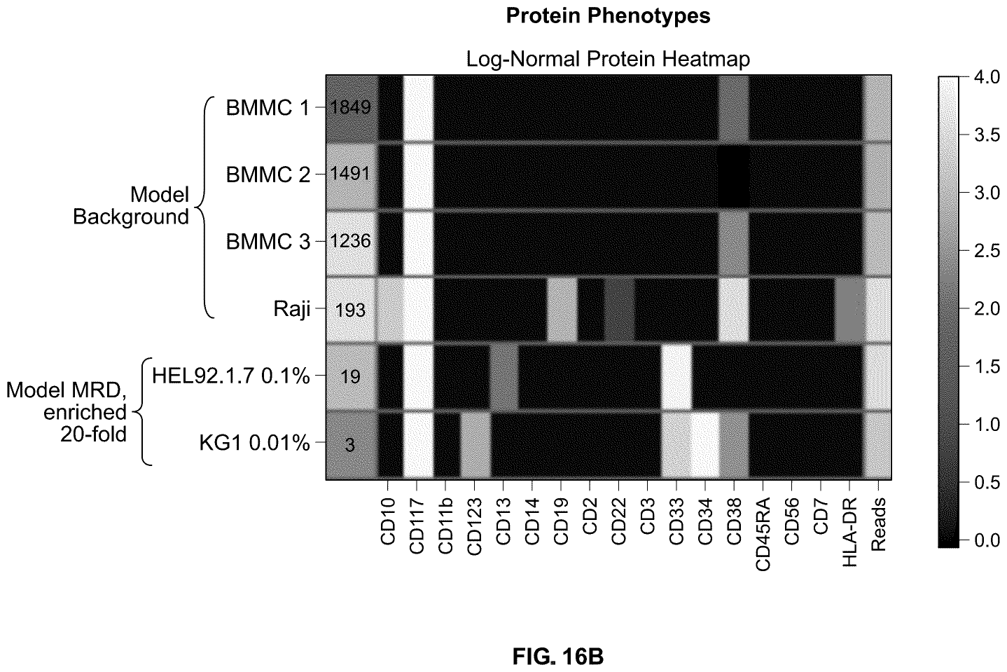

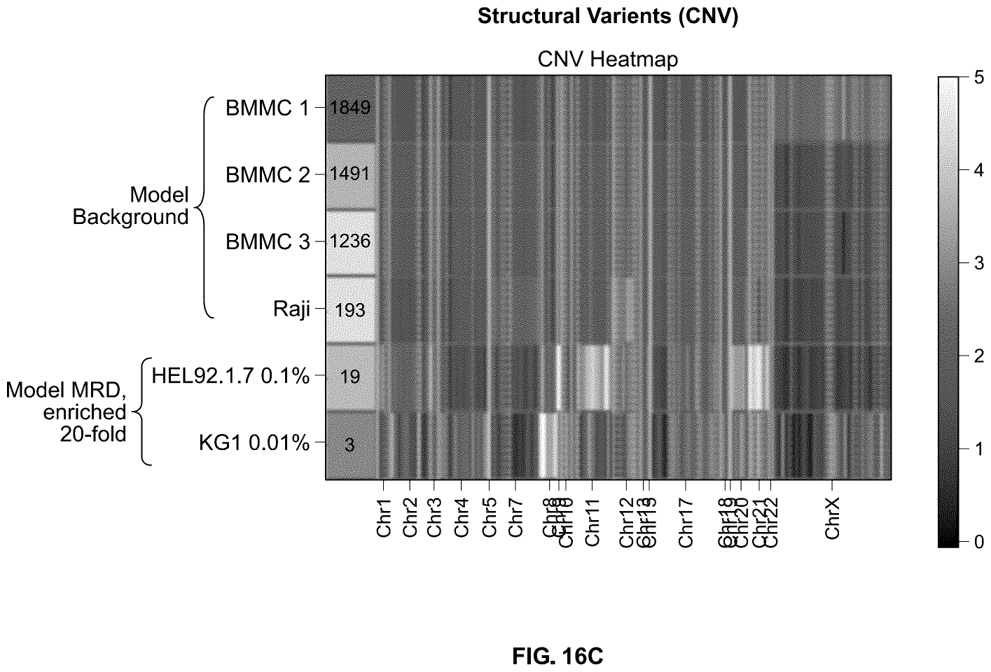

FIGS. 16A-16C depict example analysis results of MRD cells across DNA variants, protein phenotypes, and copy number variants, respectively, by implementing the methods and systems as described in FIGS. 1A, 1B, and 2-6. FIG. 16A illustrates “VAF Heatmap” for DNA variants. FIG. 16B illustrates “Log-Normal Protein Heatmap” for protein phenotypes. FIG. 16C illustrates “CNV Heatmap” for structural variants (CNV).

In this example, model MRD cells HEL92.1.7 and KG1 were enriched by 20-fold and analyzed using the systems and methods as shown in FIGS. 1A, 1B, and 2. The analysis results were compared with model background cells that were healthy cells, including BMMC 1, BMMC 1, BMMC 3, and Raji.

As shown in FIGS. 16A-16C, HEL 92.1.7 cells were detected at a sensitivity of about 0.1% against a background of healthy cells, across DNA variants, protein phenotypes, and copy number variants, respectively.

As further shown in FIGS. 16A-16C, KG1 cells were detected at a sensitivity of about 0.01% against a background of healthy cells, across DNA variants, protein phenotypes, and copy number variants, respectively.

1. A method for analyzing rare disease cells of a plurality of subjects, the method comprising:
obtaining a plurality of samples from the plurality of subjects;
for each of one or more samples in the plurality of samples, enriching the sample to obtain rare disease cells;
pooling the obtained rare disease cells across the plurality of samples;
providing the pooled rare disease cells for single-cell analysis to generate amplicons derived from analytes of the pooled rare disease cells;
sequencing the amplicons derived from analytes of the pooled rare disease cells;
clustering the rare disease cells across the plurality of samples using the sequenced amplicons; and
de-multiplexing the rare disease cells by assigning clusters of rare disease cells to individual subjects of the plurality of subjects.

2. The method of claim 1, wherein enriching the sample comprises performing any of flow cytometry, cell separation, or magnetic bead isolation.

3. The method of claim 2, wherein performing flow cytometry comprises enriching the sample for CD34+ and/or CD117+ cells.

4. The method of claim 2, wherein performing cell separation comprises providing the sample to an Angle Parsotix CTC enrichment platform.

5. The method of claim 1, wherein enriching the sample to obtain rare disease cells further comprises:
staining the rare disease cells using one or more oligo-conjugated antibodies, wherein each of the one or more oligo-conjugated antibodies are specific for a protein analyte of the rare disease cells.

6. The method of claim 1, wherein the rare disease cells are circulating tumor cells or cells informative for determining measurable residual disease (MRD).

7. The method of claim 6, wherein the method detects measurable residual disease at a sensitivity better than 0.05% or 0.01%.

8. (canceled)

9. The method of claim 6, wherein the cells informative for determining MRD are acute myeloid leukemia, myelodysplastic, or myeloid proliferative neoplasm cells.

10. The method of claim 1, wherein for each sample, enriching the sample to obtain rare disease cells comprises obtaining less than 50,000, less than 30,000, less than 500, or less than 100 rare disease cells from the sample.

11-13. (canceled)

14. The method of claim 1, wherein pooling the obtained rare disease cells across the plurality of samples comprises pooling at least 100,000 rare disease cells.

15. The method of claim 1, wherein analytes of the pooled rare disease cells are one or more of DNA, RNA, or protein analytes.

16. The method of claim 15, wherein analytes of the pooled rare disease cells are RNA analytes.

17. The method of claim 16, wherein clustering the rare disease cells across the plurality of samples using the sequenced amplicons comprises clustering the rare disease cells according to sequenced amplicons derived from the RNA analytes.

18. The method of claim 1, wherein analytes of the pooled rare disease cells comprise both DNA and protein analytes.

19. The method of claim 15, wherein clustering the rare disease cells across the plurality of samples using the sequenced amplicons comprises clustering the rare disease cells according to sequenced amplicons derived from both the DNA and protein analytes.

20. The method of claim 1, wherein the single-cell analysis comprises performing, within a droplet, cell lysis, cell barcoding, and nucleic acid amplification.

21. The method of claim 1, wherein the single-cell analysis comprises performing, cell lysis within a first droplet, and further performing cell barcoding and nucleic acid amplification in a second droplet.

22. The method of claim 1, wherein pooling the obtained rare disease cells across the plurality of samples further comprises incorporating one or more known cells derived from the plurality of subjects.

23. The method of claim 22, wherein assigning clusters of rare disease cells to individual subjects of the plurality of subjects is based on presence of the one or more known cells within the clusters.

24. A system for analyzing rare disease cells of a plurality of subjects, the system comprising:
an enrichment platform for enriching a plurality of samples obtained from the plurality of subjects to obtain rare disease cells;
a single-cell analysis platform for generating amplicons, wherein the amplicons are derived from analytes of the rare disease cells pooled across the plurality of samples;
a sequencing platform for sequencing the amplicons derived from analytes of the pooled rare disease cells; and
a computing device for clustering by using the sequenced amplicons and de-multiplexing the rare disease cells by assigning clusters of rare disease cells to individual subjects of the plurality of subjects.

25-46. (canceled)
